(19) United States
(12) Patent Application Publication
(10) Pub. No.: US 2024/0376133 A9
(43) Pub. Date: Nov. 14, 2024

(54) CROSSLINKING COMONOMERS FOR HIGH PERFORMANCE DEGRADABLE THERMOSETS

(71) Applicant: Massachusetts Institute of Technology, Cambridge, MA (US)

(72) Inventors: Jeremiah A. Johnson, Boston, MA (US); Peyton Shieh, Cambridge, MA (US); Keith Husted, Boston, MA (US)

(73) Assignee: Massachusetts Institute of Technology, Cambridge, MA (US)

(21) Appl. No.: 18/035,922

(22) PCT Filed: Nov. 9, 2021
(86) PCT No.: PCT/US2021/058668

§ 371 Date: May 8, 2023

Related U.S. Application Data
(60) Provisional application No. 63/111,608, filed on Nov. 9, 2020.

(51) Int. Cl. C07F 7/18; C07F 7/02; C08G 65/26

(52) U.S. Cl. CPC C07F 7/1804; C07F 7/025; C08G 65/2642

(57) ABSTRACT

The present disclosure provides compounds of the formula (I): The present disclosure also provides copolymers prepared by polymerizing a first monomer (e.g., dicyclopentadiene) and the compounds. The copolymers may show increased degradability and increased or maintained glass-transition temperature, as compared to homopolymers of the first monomer.

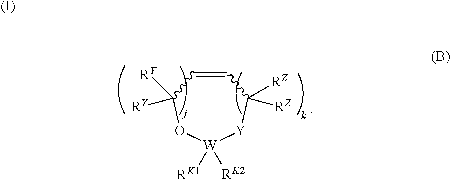

### RELATED APPLICATIONS

This application claims priority under 35 U.S.C. § 119(e) to U.S. Provisional Application No. 63/111,608, filed Nov. 9, 2020, which is incorporated herein by reference.

### SUMMARY OF THE DISCLOSURE

There is a need to improve the reprocessability of thermosets. An approach to convert existing thermosets into degradable variants would involve the use of a low-cost co-monomer additive that, when introduced at low levels during standard thermoset formulation conditions, could introduce cleavable bonds at precise locations within the thermoset polymer network enabling material degradation with otherwise little to no impact on properties. The use of such co-monomer strategies to imbue commodity polymers with degradability or reprocessahility is exceedingly rare. To our knowledge, such an approach has not been demonstrated in the context of existing high-performance thermosets.

Here, in one aspect, we establish this co-monomer approach in the context of commercially important thermosets, such as poly-dicyclopentadiene (pDCPD). pDCPD may be prepared through ring-opening metathesis polymerization (RONTP) of the abundant hydrocarbon feedstock dicyclopentadiene (DCPD). See, e.g., U.S. patent application Ser. Nos. 16/542,824, filed Aug. 16, 2019, and 17/022,021, filed Sep. 15, 2020, which are incorporated herein by reference. In this curing process, the norbornene component of DCPD polymerizes rapidly to produce linear polymer strands that are subsequently crosslinked through metathesis reactions of their cyclopentene sidechains. The resulting entirely hydrocarbon thermoset is valued for its high impact resistance and compatibility with reaction injection molding processes. Moreover, emerging manufacturing concepts, such as frontal polymerization, enable pDCPD curing with energy consumption orders-of-magnitude lower than other thermosets (e.g., epoxies).

The present disclosure provides, for example, compounds, copolymers, hydroxylated oligomers, hydroxylated polymers, conjugates, compositions, kits, methods of preparing the compounds, methods of preparing the hydroxylated oligomers and hydroxylated polymers, methods of preparing the copolymers, and methods of preparing the conjugates.

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiorner, diastereomer or geometric isomer, or can be in the form of a mixture of stereoisomers, including racemic mixtures and mixtures enriched in one or More stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistty of Carbon Compounds (McGraw-Hill, NY, 1962); and Wilen, Tables of Resolving Agents and Optical Resolutions p. 268 (E. L, Eliel, Ed., Univ. of Notre Dame Press, Notre Dame, IN 1972). The present disclosure additionally encompasses compounds described herein as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic straight chain unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” is intended herein to include alkyl, alkenyl, alkynyl, cycloa41, cycloalkenyl, and cycloalkynyl moieties.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆, alkyl.

“Alkyl” refers to a radical of a straight-chain or branched saturated hydrocarbon group having from 1 to 20 carbon atoms (“C₁₋₂₀, alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄alkyl”). In some embodiments, an alkyl group has 1 to 3 carbon atoms (“C₁₋₃ alkyl”). In some embodiments, an alkyl group has 1 to 2 carbon atoms (“C₁₋₂alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec; butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu. e.g., unsubstituted n-butyl (n-Bu), unsubstituted tert-butvl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ≡CH). The moieties =CH₂ and ≡CH are also alkyl.

In some embodiments, an alkyl group is substituted with one or more halogens. “Perhaloalkyl” is a substituted alkyl group as defined herein wherein all of the hydrogen atoms are independently replaced. by a halogen, e.g., fluoro, brotno, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, amd the like.

“Alkenyl” refers to a radical of a straight-chain or branched hydrocarbon group having from 2 to 20 carbon atoms, one or more two, three, or four, as valency permits) carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₈), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified

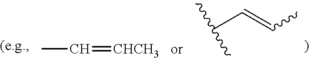

may be in the (L)- or (Z)-configuration.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”), In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (CO₁₀), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted. C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valen.cy permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C=C triple bonds in the carbocyclic ring is referred to as “cycloalkynyl.” “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. in certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic,

In some embodiments, “carbocyclyl” is a monocyclic, saturated carbocyclyl group having from 3 to 10 ring carbon atoms (“C₃₋₁₀ cycloalkyl”), in some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”), In some embodiments, a cycloalkyl group has 5 to 6 ring carbon atoms (“C₅₋₆ cycloalkyl”). in some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₃). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted. (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cyclloalkyl.

“Beterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, sulfur, or silicon (“3-13 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more e.g., two, three, or four, as valency permits) double bonds in all the rings of the heterocyclic ring system that are not aromatic or heteroaromatic. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 310 embered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, sulfur, and silicon (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, sulfur, and silicon (“5-6 membered heterocyclyl ”). In some embodiments, the 5-6 membered heterocyclyl has 1-3 ring heteroatoms selected from nitrogen, oxygen, sulfur, and silicon. In some embodiments, the 5---6 membered heterocyclyl has 1-2 ring heteroatoms selected from nitrogen, oxygen, sulfur, and silicon, In some embodiments, the 5-6 membered heterocyclyl has one ring heteroatom selected from nitrogen, oxygen, sulfur, and silicon.

Exemplary 3-membered heterocyclyl groups containing one heteroatom include azirdinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5—membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrohenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquincilinyl, and the like.

“Aryl” refers to a radical of a monocyclic or polycyclic bicyclic or tricyclic) 4n+2 aromatic ring system (e.g., having 6, 10, or π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”, naphthyl such as 1-naphthyl and 2-naphthyl). In some embodiments, an aryl group has fourteen ring carbon atoms (“C₁₄ aryl”; e.g., anthracyl). “Amyl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents, In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl , quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroatyl group is a 5-10 membered aromatic ring system having ring carbon atoms and 1-4 ring heteroatorns provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic. heteroaryl groups include indolyl, isoindolyl, indazolyl, benzotriazolyl, benzothiophenyl, isobeazothiophenyl, benzofuranyl, benzoi sofuranyl, benzimi dazolyl, benzoxazolyl, benzisoxazolyl, benzoxadiazolyi, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (cg., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). in general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN , —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aap}, —NR^{bb}C(═NR^{bb})N(R^{bb})₂, —C(═O)NR^{bb}SO₃R^{aa}, —NR^{bb}SO₂R^{aa}, —SO₂N(R^{bb})₂, —SO₂R^{bb}), —SO₂R^{aa}, —OSO₂R^{aa}, —S(═O)R^{aa}, —OS(═O)R^{aa}, —Si(R^{aa})₃, —C(═S)N(R^{bb})₂, —C(═O)SR^{aa}, —C(═S(SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(T^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃, —P(OR^{cc})₃⁺X⁻, —P(R^{ccpk )}₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc},

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₁₋₁₀alkenyl, heteroC₁₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂)R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{dd})₂, —P(═O)(OR^{cc})₂, —P(═O)(N(R^{cc})₂)₂, —C₁₋₁₀ alkyl, C₁₋₁₀ perhaloalkyl, C₂₋₂₀ alkenyl, C₂₋₁₀ alkynyl, heteroCi₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion,

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{cc})R^{ff}, —SH, —SR^{cc}, —SSR^{cc}, —C(═O)R^{cc}, —CO₂H, —CO₂R^{cc}, —OC(═O)R^{cc}, —O(CO₂R^{cc}, —C(═O)N(R^{ff})₂, —OC(═O)N(R^{ff})₂, —NR^{ff}C(═O)R^{cc}, —NR^{ff}CO₂R^{cc}, —NR^{ff}C(═O)R^{cc}, —NR^{ff}C(═O)N)R^{ff})₂, —C(C═NR^{ff})OR^{cc}, —OC(═NR^{ff})R^{cc}, —OC(═NR^{ff})OR^{cc}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{cc}, —SO₂N(R^{ff})₂, —SO₂R^{cc}, —SO₂OR^{cc}, —OSO₂R^{cc}, —S(═O)R^{cc}, —Si(R^{cc})₃, —OSi(R^{cc}), —C(—S)N(R^{ff})₂, —C(═O)SR^{cc}, —C(═S)SR^{cc}, —SC(═S)SR^{cc}, —P(═O)(OR^{cc})₂, —P(═O)(OR^{cc}₂, —P(═O)(R^{cc})₂, —OP(═O)(R^{cc})₂, —C(═O)SR^{cc}, —C(═S)SR^{cc}, —SC(═S)SR^{cc}, —P(═O)(OR^{cc})₂, —P(═O)(R^{cc}), —OP(═O)(R^{cc})₂, —OP(═O)(OR^{cc})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocydyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{cc} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆ alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkvnyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆ alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN , —NO₂, —N₃, —SO₂H, —SO₃H, —OH, —OC₁₋₆ alkyl, —ON(C₁₋₆ alkyl)₂, —N₁₋₆ alkyl)₂, —N(C₁₋₆ alkyl)₃⁺X⁻, —NH(C₁₋₆ alkyl)₂⁺X⁻, NH₂(C₁₋₆ alkyl)⁺X⁻, —NH₃⁺X⁻, —N(OC₁₋₆ alkyl)(C₁₋₆ alkyl, ₁₋₆ N(OH)(C₁₋₆ alkyl), ₁₋₆ NH(OH), —SH, —SC₁₋₆ alkyl, —SS(C₁₋₆ alkyl), —C(═O)(₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl, —NHCO₂(₁₋₆ alkyl), —NHC(═O)N(C₁₋₆ alkyl)₂, —MHC(═O)NH(C₁₋₆ alkyl), —NHC(═O)NH₂, —C(═NH)O(C₁₋₆ alkyl), —OC(═NH)(C₁₋₆ alkyl, —OC(═MH)OC₁₋₆ alklyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —PC(NH(C₁₋₆ alkyl, —OC(NH)NH₂, —NHC(NH)N(C₁₋₆ alkyl₂, —NHC(═NH)NH₂, —NHSO₂(C₁₋₆ alkyl), —SO₂N(C₁₋₆ alkyl)₂, —SO₂NH(C₁₋₆ a;ly;), —SO₂MNH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl(₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heterpC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S, wherein X⁻ is a counterion.

In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —(C(═O)R^{aa}, —OCO₂, R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylinethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylinethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counted ons include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesullfonate, benzenesulfonate, 10-camphor sulfonate, naphthalene 2 sulfonate, naphthalene -1-sulfonic acid 5-sulfonate, ethan-1-sulfonic acid 2-sulfonate, and the like), carboxylate ions (e,g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., VB₁₁H₁₂⁻or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{aa})₂, —CN, —C(═O)R^{aa}, —C(═O)N(R^{cc})₂, —CO₂R^{aa}, 13 SO₂R^{aa}, —C(═NR^{aa})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc}(₃, —C(═O)SR^{cc}, —C(═S)SR^{cc}, —P(═O)(OR^{cc})₂, —P(═O(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroatkynyl, carbocyclyl, heterocyclyl, aryl, and heteroaryl is independently substituted with 0, 1, 2, 3, 4, or 5 R^{aa} groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen. substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})PR^{aa}, —C(═NR^{cc})N(R^{cc})₂, —SO₂N(R^{cc})₂, —SO₂R^{cc}, —SO₂OR^{cc}, —SOR^{aa}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include foonamide, acetamide, chloroacetamide, tfichloroacetamide, nifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyppropanamide, 2-methyl-2(o-nitrophenoxy)propanamide, 2-methyl-2-(o-phenylazophenoxy)propanamide, 4-chlorobutanamide, 3-methyl 3-nitrobutanamide, o-nitrocinnamide,N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamate, 9-fluorenylmethyl carbamate (Fmoc), 9-(2-sulfo)fluorenyhnethyl carbamate, 9--(2,7-dibromo)fluoroenylmethyl carbamate, 2,7-di-t-butyl-[9-(10,10-dioxo--10,10,10,10-tetrahydrothioxanthyl]methyl carbamate (DBD-Tmoc), 4-methoxyphena.cyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2 drbromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridypethyl carbamate (Pyoc), 2-(N,N-dicyclohexylearboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coe), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorohenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate. 2-methylsulfonylethyl carbamate, 2-(p-toluenesulfonyl)ethyl carbamate, [2-(1,3-dithianypl)]methyl carbamate (Dmoc), 4-methylthiophenyl carbamate (Mtpc), 2,4-dimethytthiophenyl carbamate (Bmpc), 2-phosphonioethyl carbamate (Peoc), 2-triphenylphosphontoisopropyl carbamate (Ppoc), 1,1-dimethyl-2-cyanoethyl carbamate, m-chloro-p-acyloxybenzyl carbamate:, p-(dihydroxyboryl)benzyl carbamate, 5-benzisoxazolylmethyt carbamate, 2-(trifluoromethyl)-6-chromonylmethyl carbamate (Tcroc), m-nitrophenyl carbamate, 3,5-dimethoxybenzyl. carbamate, o-nitrobenzyi carbamate, 3,4-dimethoxy 6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethyllcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoboryni carbamate, isobutyl carbamate, isomicotinyl carbamate, p-(p′-methoxyphenylazo)benzyl carbamate, 1-methylcyclobutyl carbamate, 1-methylcyclohexyl carbamate, 1-methylcyclopropylmethyl carbamate, 1-methyl-1(3,5-dimethoxyphenypethyl)ethyl carbamate, 1-methyl-1-(pphenylazophenyl)ethyl carbamate, 1-methyl 1 phenylethyl carbamate, I methyl I (4 pyridyl)ethyl carbamate, phenyl carbamate,p-(phenylazo)benzyl carbamate, 2,4,6 tri t butylphenvl carbamate, 4-(trimethylammonium)benzyl carbamate, and 2,4,6-trimethylbenzyl carbamate.

Sulfonamide nitrogen protecting groups (e.g., -S(=0) 2 Raa) include p-toluenesulfonamide (Ts), benzenesulfonamide, 2,3,6,-trimethyl 4 -methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl 4 methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman 6 sulfonamide (Pmc), methanesulfonamide (Ms), βtrimethylsilytethanesuifonamide (SES), 9-anthracenesulfonamide, (4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-2-(trimethylsilypethoxyjimethylamine (SEM), N-3-acetoxypropylamine, N-(1-isopropyl 4-nitro-2-oxo 3-pyrrolin 3-yl)amine, quaternary ammonium salts, N-benzylamine, N-di(4-methoxyphenyl)methylamine, N-5-dibenzosuberylamine, N-tiiphenylinethylamine (Tr), N-[(4-methoxyphenyl)diphenylmethyl]amine (MMTr), N-9-phenylfluorenyiamine (PhF), N-2-7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenyhnethyleneamine, N-[(2-pyridyl)mesityl]methyleneamine, N-(N′,N′-dimethylaminomethylene)amine, N,M′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchmniuin- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophospamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro 4-methoxybenzenesulfenamide, triphenylmethylsulfenamide, and 3-nitropytidinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, in certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted. C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃^{'}X⁻, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), i-butylthiomethyl, (phenyldimethylsilypmethoxymethyl (SMOM), benzyloxymethyl (BC)M),p-methoxybenzyloxymethyl (PMBM), (4-inethoxyphenoxy)methyl (p-AOM), guaiacolmethyl (GUM), i-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilypethoxymethyl (SEMOR), tetrahydropyranyl (THP), 3-bromotetrahydropyranyl, tetrahydrothiopyranyl, 1-methoxycyclohexyl, 4-methoxytetrahydropyra.nyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl(CIMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2 chloroethoxy)ethyl, 1-methyl-1-methoxyethyl, 1methyl-1-benzyloxyethyl, 1-methyl 1-benzyloxy 2-fluoroethyl, 2,2,2-trichloroethyl, 2-trimethylsilylethyl, 2-(phenylselenyl)ethyl, i-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-pieolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl,p-methoxyphenyldiphertylmethyl, di(p-methoxyphenyl)plienylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-diinethoxyphenyl)methyl, 1,1-bis(4-methoxyphenyI)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyi S,S-dioxido, trimethylsilyl (TMS), triethylsilyl (TEs), triisopropylsilyl (TIPS), dimethylisopropylsilyl (IPDMS), diethylisopropylsilyl (DEIPS), dimethylthexylsilyl, t-butyldimethylsilyl (TBDMS), i-butyldiphenylsilyl (TBDPS), tribenzylsilyl, tri-p-xylyisilyl, triphenylsilyi, diphenylmethylsilyl (DPMS), t-butylrnethoxyphenyisilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenyhnethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimetboxybenzyl carbonate, alkyl o-anitrobenzyl carbonate, alkyl p-nitrobenzyl carbonate, alkyl may-benzyl thiocarbonate, 4-ethoxy-1-naphthyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethyibutyl)phenoxy acetate, 2,4-bis(1,1-dimethylpropyl)phenoxyacetate, ehlorodiphenylacetate, isobutyrate, monosuccinoate, (E)-2-methyl-2-butenoate, o-(methoxyacypbenzoate, α-naphthoate, nitrate, alkyl N,N,N′,N′-tetramethylphosphorodiamidate, alkyl N-phenylcarbamate, borate, dimethylphosphinothioyl, alkyl 2,4--dinitrophenylsulfenate, sulfate, methanesulfonate (mesylate), ben sulfonate, and tosylate (Is).

In certain embodiments, an oxygen protecting group is silyl, TBDPS, TBDMS, TIPS. TES, TMS, MOM, THP, t-Bu, Bn, allyl, acetyl, pivaloyl, or benzoyl.

In certain embodiments, the sulfur atom substituents are independently substitute((e.g., substituted with one or more halogen) or unsubstituted C₁₋₆ alkyl, —C(═O)R^{aa}, —CO₂R^{aa}, C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, ;13 C(═NR^{bb})R^{aa}, —C(═NR^{bb})OR^{aa}, —C(═N^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P9═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3r d edition, John Wiley K. Sons. 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by subtracting the atomic weight of a hydrogen atom from the molecular weight of the molecule R—H. The “molecular weight” of -L-, wherein -L- is any divalent moiety, is calculated by subtracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 &lot, In certain embodiments, a. substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

The term “leaving group” is given its ordinary meaning in the art of synthetic organic chemistry and refers to an atom or a group capable of being displaced by a nucleophile. Examples of suitable leaving groups include halogen (such as F, Cl, Br, or I (iodine)), alkoxycarbonyloxy, aryl oxycarbonyloxy, alkanesulfonyloxy, arenesulfonyloxy, alkylcarbonyloxy (e.g., acetoxy), arylcarbonyloxy, aryloxy, methoxy, N,O-dimethylhydroxylamino, pixyl, and halloformates. In some cases, the leaving group is a sulfonic acid ester, such as toluenesulfonate (tosylate, -OTs), methanesulfonate (mesylate, OMs), p-bromobenzenesulfonyloxy (brosylate, -OBs), -OS(═O)₂(CF₂)₃CF₃ (nonaflate, -ONO, or trifluoromethanesulfonate (triflate, -OTf). in some cases, the leaving group is a brosylate, such asp-bromobenzenesulfon.yloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other examples of leaving groups are water, ammonia, alcohols, ether moieties, thioether moieties, zinc halides, magnesium moieties, diazonium salts, and copper moieties.

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic, and organic acids and bases. Examples of acid addition salts are salts of an amino group formed with inorganic acids such as hydrochloric acid, hydrobromic acid, phosphoric acid, sulfuric acid, and perchloric acid, or with organic acids such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, heinisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, mtoluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

“Compounds” include, e.g., small molecules and macromolecules. Macromolecules include, e.g, polymers, peptides, proteins, carbohydrates, monosaccharides, oligosaccharides, polysaccharides, nucleoproteins, mucoproteins, lipoproteins, synthetic polypeptides or proteins, glycoproteins, steroids, nucleic acids, DNAs, RNAs, nucleotides, nucleosides, oligonucleotides, antisense oligonucleotides, lipids, hormones, vitamins, and cells.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 gimol, not more than 900 gimol, not more than 8(X) g/mol, not more than 700 glinol, not more than 600 g/mol, not more than 500 g/mol, not more than 400 g/mol, not more than 300 g/mol, not more than 200 g/mol, or not more than 100 g/inol. In certain embodiments, the molecular weight of a small molecule is at least 100 g/mol, at least 200 g/triol, at least 300 g/mol, at least 400 g/mol, at least 500 g/mol, at least 600 glinol, at least 700 g/mol, at least 800 glmol, or at least 900 g/mol, or at least 1,000 g/mol. Combinations of the above ranges (e.g., at least 200 g/mol and not more than 500 glmol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 2.1 C.F.R. §§ 500 through 589, incorporated herein by reference. All listed drugs are considered acceptable for use in accordance with the present disclosure.

The term “oligomer” refers to a compound comprising two to ten, inclusive, covalently connected repeating units. In certain embodiments, an oligomer comprises two to five, inclusive, covalently connected repeating units. In certain embodiments, an oligomer comprises six to ten, inclusive, covalently connected repeating units.

The term “polymer” refers to a compound comprising eleven or more covalently connected repeating units. In certain embodiments, a polymer is naturally occurring. In certain embodiments, a polymer is synthetic (e.g., not naturally occurring). In certain embodiments, the Mw of a polymer is between 1,000 and 2,000, between 2,000 and 10,000, between 10,000 and between 30,000 and 100,000, between 100,000 and 300,000, between 300,000 and 1,000,000, Ono', inclusive. In certain embodiments, the Mw of a polymer is between 2,000 and 1,000,000, g/mol, inclusive.

When a polymer is prepared by polymerizing two or more different types of monomers, the monomers may be referred to as comonomers. The polymer may be referred to as a copolymer.

The term “average molecular weight” may encompass the number average molecular weight (Mₙ), weight average molecular weight (M_{w}), higher average molecular weight (M_{z} or M_{z} +1), GPC/SEC (gel permeation chromatography/size-exclusion chromatography)-determined average molecular weight (Mₚ), and viscosity average molecular weight (Mᵥ). Average molecular weight may also refer to average molecular weight as determined by gel permeation chromatography.

The term “degree of polymerization” (DP) refers to the number of repeating units in a polymer. In certain embodiments, the DP is determined by a chromatographic method, such as gel permeation chromatography. For a homopolymer, the DP refers to the number of repeating units included in the homopolymer. For a copolymer of two types of monomers (e.g., a first monomer and a second monomer) wherein the molar ratio of the two types of monomers is about 1:1, the DP refers to the number of repeating units of either one of the two type of monomers included in the copolymer. For a copolymer of two types of monomers (e.g., a first monomer and a second monomer) wherein the molar ratio of the two types of monomers is not about 1:1, two DPs may be used. A first DP refers to the number of repeating units of the first monomer included in the copolymer, and a second DP refers to the number of repeating units of the second monomer included in the copolymer. Unless provided otherwise, a DP of “xx”, wherein xx is an integer, refers to the number of repeating units of either one of the two types of monomers of a copolymer of two types of monomers (e.g., a first monomer and a second monomer) wherein the molar ratio of the two types of monomers is about 1:1, Unless provided otherwise, a DP of “xx-yy”, wherein xx and yy are integers, refers to xx being the number of repeating units of the first monomer, and yy being the number of repeating units of the second monomer, of a copolymer of two types of monomers (e.g., a first monomer and a second monomer) wherein the molar ratio of the two types of monomers is not about 1:1.

The term “ring-opening metathesis polymerization (ROMP)” refers to a type of olefin metathesis chain-growth polymerization that is driven by the relief of ring strain in cyclic olefins (e.g norbornene or cyclopentene). The catalysts used in the ROMP reaction (“metathesis catalyst”) include RuCl₃/alcohol mixture, bis(cyclopentadienyl)dimethylzirconium(IV), dichloro[1,3-bis(2,6-isopropylphenyl)-2-imidazolidinylidene](benzylidene)(tricyclohexylphosphine)ruthenium(II), dichloro[1,3-Bis(2-methylphertyl)-2-imidazolidinylidene](benzylidene)(tricyclohexylphosphine) ruthenium(II), dichloro[1,3-bis(2,4,6-trimethylphenyl)-2-imidazolidinylidene][3-(2-pyridinyl)propylidene]ruthenium(II), dichloro(3-methyl-2-butenylidene)bis (tricyclopentylphosphine)ruthenium(II), dichloro[1,3-bis(2-methylphanyl)-2-imidazolidinylidene](2-isopropoxyphenylmethylene)ruthenium(II) (Grubbs C571), dichloro(benzylidene)bis(tricyclohexylphosphine)tuthenium(II) (Grubbs I), dichloro[1,3-bis(2,4,6-trimethylphenyl)-2-imidazolidinylidene](benzylidene)(tricyclohexylphosphine) ruthenium(II). (Grubbs II), and dichloro[1,3-bis(2,4,6-trimethylphenyl)-2-imidazolidinylidene](benzylidene)bis(3-bromopyridine)ruthenium(H) (Grubbs III).

The terms “v/y” and “v:v” refer to volume per volume and is used herein to express concentrations of monomers. Unless otherwise provided, a percent concentration of a second monomer in a first monomer is expressed in v/v. For example, a mixture of a first monomer and 10% second monomer refers to a mixture of a first monomer and a second monomer, wherein the volume of the second monomer is 10% of the combined volumns of the first and second monomers.

The disclosure is not intended to be limited in any manner by the above exemplary listing of substituents. Additional terms may be defined in other sections of this disclosure.

### BRIEF DESCRIPTION OF THE DRAWINGS

The figures are exemplary and do not limit the scope of the present disclosure.

FIG. 1A shows crosslinking comon.omers for preparing DCPD copolymers (pDCPDs; copolymers prepared by polymerizing DCPD and one or more different types of monomers).

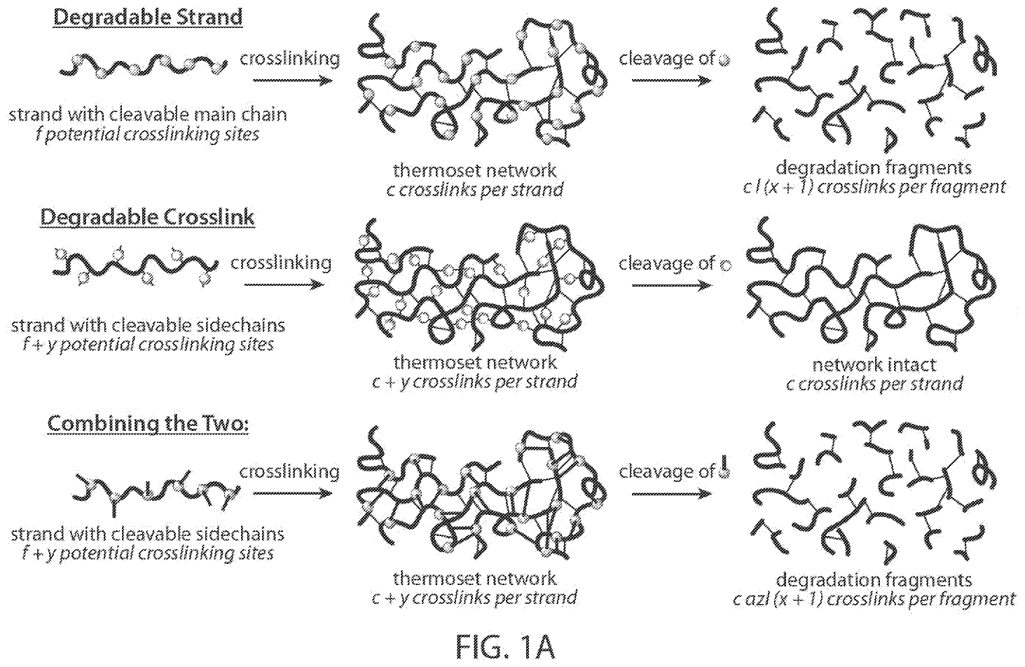

FIG. 1B shows the structures of certain comparative monomers.

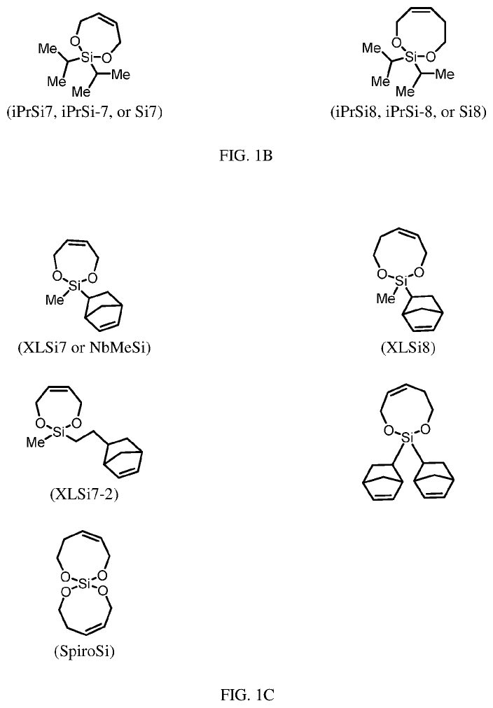

FIG. 1C shows the structures of certain monomers of the present disclosure.

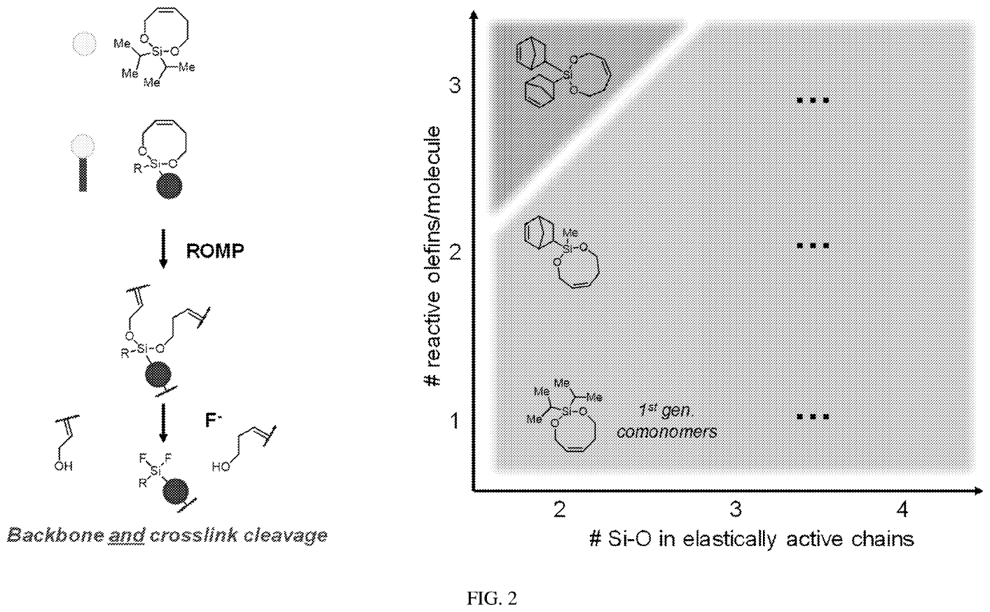

FIG. 2 shows the design of crosslinking comonomers.

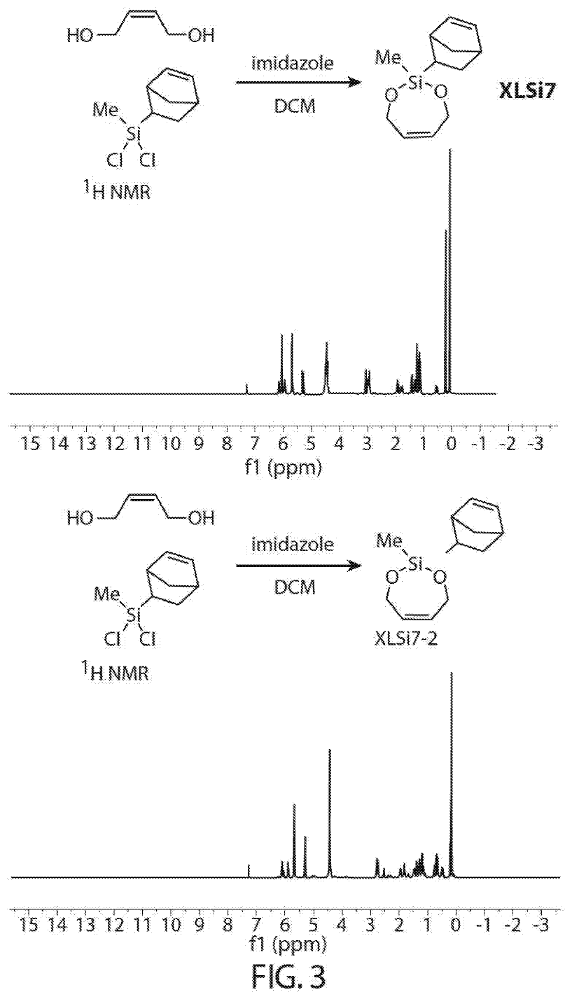

FIG. 3 shows ¹H NMRs of crosslinked silyl ether monomers. Top panel: XLSi7. Bottom panel: XLSi7-2.

FIG. 4A shows an exemplary degradation of copolymers of the present disclosure. Before degradation, the vial on the left contained a DCPD-XLSi7 copolymer, and the vial on the right contained a DCPD-XLSi7-2 copolymer.

FIG. 4B shows fluorine incorporation in pDCPD fragments. Left panel: ¹H NMR. Right panel: ¹⁹F NMR.

FIGS. 5A to 5C show mechanical characterization of DCPD-XLSi7 copolymers.

FIG. 6 shows the glass-transition temperature (T_{g}) of DCPD copolymers with a crosslinking monomer (XL, e.g., XLSi7) or a non-crosslinking monomer (e.g., Si8 or Si7).

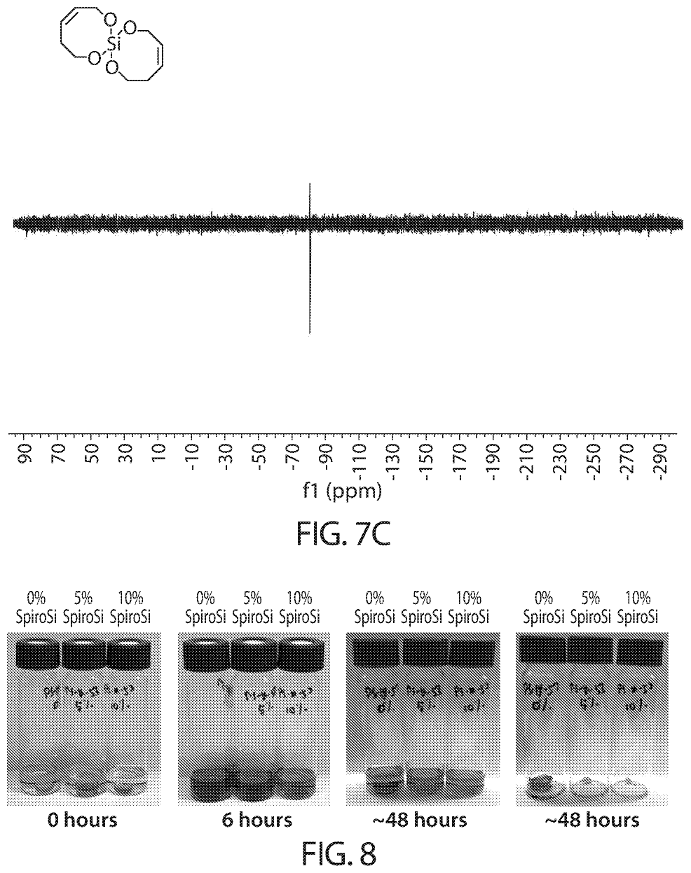

FIGS. 7A to 7C show ¹H NMR (FIG. 7A), ¹³C NMR (FIG. 7B) and ²⁹ Si NMR (FIG. 7C) of the spirocyclic monomer SpiroSi.

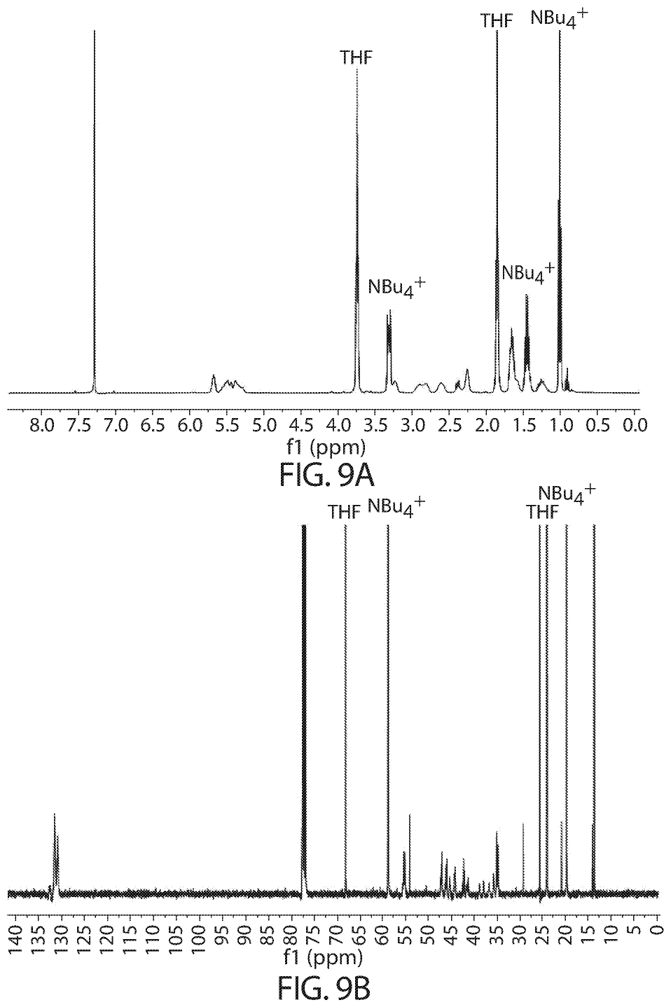

FIG. 8 shows exemplary results of 200 mg samples of DCPD-SpiroSi copolymers containing 0%, 5%, or 10% (v:v) of SpiroSi after being treated with 5 mL of 0.2 M TBAF in THF.

FIGS. 9A to 913 show a ¹H NMR spectrum (FIG. 9A) and a ¹³C NMR spectrum (FIG. 9B) of the soluble materials obtained from a degradation of a DCPD-SpiroSi copolymer containing 5% (v:v) of SpiroSi.

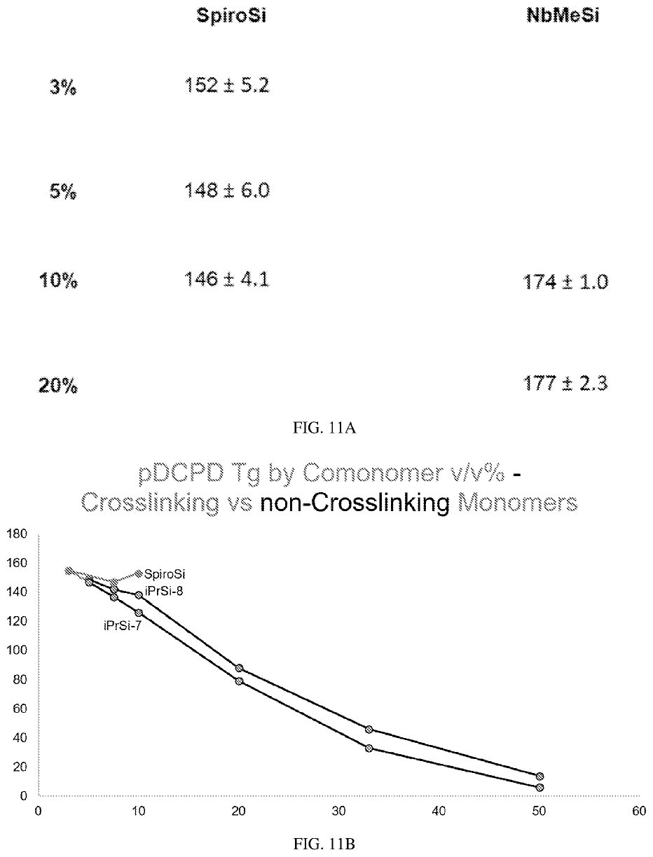

FIGS. 110A to 10B show a NMR spectrum (FIG. 10A) and a ¹³ C NMR spectrum (FIG. 10B of NbMeSi.

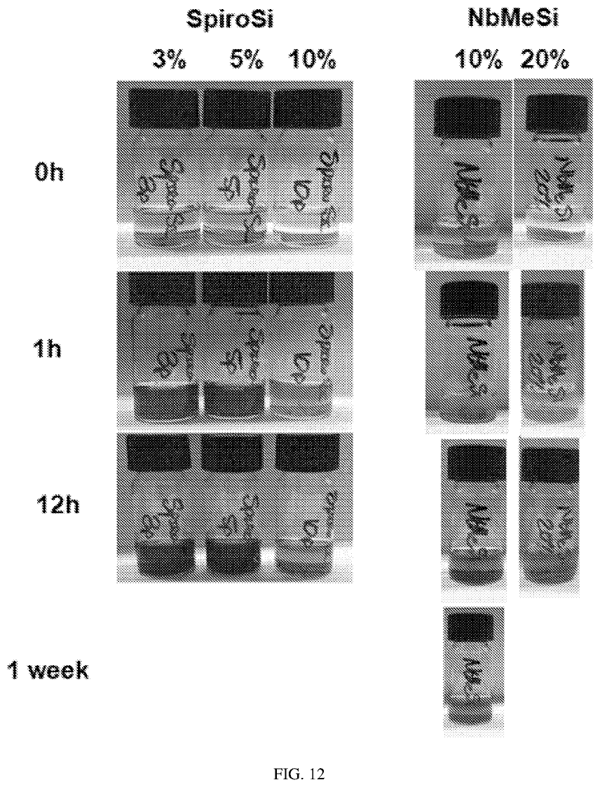

FIGS. 11A to 11B show the dependence of the copolymer's T_{g} on the identityand loading of the crosslinking and non-crosslinking comonomers. T_{g} of native pDCPD was determined to be 166 Celsius. Tan(δ) was chosen as global maximum value across curve. Crosslinking comonomers maintained desired degradability and also maintained (e.g., SpiroSi) or even boosted (e.g., NbMeSi) desirable thermal properties, whereas non-crosslinking comonomers (e.g., iPrSi-8, iPrSi-7) diminished thermal properties.

FIG. 12 shows exemplary degradations of DCPD-SprioSi copolymers containing 3%, 5%, or 10% (v:v) of SpiroSi (left panel) and exemplary degradations of DCPD-NbMeSi copolymers containing 10% or 20% (v:v) of NbMeSi (right panel).

### DETAILED DESCRIPTION OF CERTAIN EMBODIMENTS OF THE DISCLOSURE

The present disclosure provides the subject matter described herein, e.g., subject matter described in the claims. For example, the present disclosure provides compounds, copolymers, hydroxylated oligomers, hydroxylated polymers, conjugates, compositions, kits, methods of preparing the compounds, methods of preparing the hydroxylated oligomers and hydroxylated polymers, methods of preparing the copolymers, and methods of preparing the conjugates.

The compounds may be useful for preparing the copolymers, In certain embodiments, the compounds are second monomers (second comonomers) for preparing the copolymers. In certain embodiments, the first monomers (first comonomers) are dicyclopenta.diene (DCPD). Ring opening metathesis polymerization (ROMP) may be employed, in the presence of a metathesis catalyst, to prepare the copolymers. The copolymers may be thermosetting polymers. Thermosetting polymers are typically difficult to be recycled. The copolymers may be degradable (e.g., biodegradable). In certain embodiments, one or more O—Si or O—C bonds of the copolymers are the degradation sites. In certain embodiments, the presence of the second monomers in the preparation of the copolymers increase the degradability of the copolymers.

It may be desirable to increase or maintain the glass-transition temperature (T_{g}) of the copolymers while the degradability of the copolymers is increased. In certain embodiments, the presence of the second monomers in the preparation of the copolymers increase the T_{g} of the copolymers. In certain embodiments, the presence of the second monomers in the preparation of the copolymers do not significantly decrease the T_{g} of the copolymers. In certain embodiments, the second monomers are crosslinking monomers, e.g., monomers with two or more polymerization handles (e.g., ROMP handles). in certain embodiments, the presence of the crosslinking monomers in the preparation of the copolymers increase the T. of the copolymers. In certain embodiments, the presence of the crosslinking monomers in the preparation of the copolymers do not significantly decrease the T_{g} of the copolymers.

The hydroxylated oligomers and hydroxylated polymers may be degradation (e.g., hydrolysis) products of the copolymers. The hydroxylated oligomers and hydroxylated polymers may be soluble in, e.g., commercially available solvents (e.g., THF). The hydroxylated oligomers and hydroxylated polymers may be useful for recycling the copolymers. The hydroxylated oligomers and hydroxylated polymers may be useful as starting materials for preparing additional oligomers or polymers.

In one aspect, the present disclosure provides compounds of Formula (B):

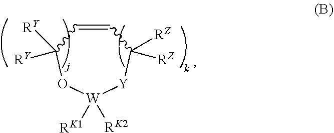

and salts thereof, wherein:

W is carbon or silicon;

Y is O or C(R^{Q})₂;

each instance of R^{Q} is independently hydrogen, halogen, or substituted or unsubstituted, C₁₋₆ alkyl;

each instance of R^{Y} is independently hydrogen, halogen, or substituted or unsubstituted, C₁₋₆ alkyl;

each instance of R^{Z} is independently hydrogen, halogen, or substituted or unsubstituted, C₁₋₆ alkyl;

R^{K1} is hydrogen, halogen, substituted or unsubstituted, C₁₋₁₀ alkyl, substituted or unsubstituted, C₂₋₁₀ alkenyl, substituted or unsubstituted, C₂₋₁₀alkynyl, substituted or unsubstituted carbocyclyl, substituted or unsubstituted heterocyclyl, substituted or unsubstituted aryl, substituted or unsubstituted heteroaryl, L^{K1}-(substituted or unsubstituted carbocyclyl),-L^{K1}-(substituted or unsubstituted heterocyclyl), -L^{K1}-(substituted or unsubstituted aryl), 1L^{K1}-(substituted or unsubstituted heteroaryl), or —OR^{N1};

L^{K1} is —O—, substituted substituted or unsubstituted, C₁₋₁₀ alkylene, substituted or unsubstituted, C₂₋₁₀ heteroalkylene, substituted or unsubstituted carbocyclyiene, substituted or unsubstituted heterocyclylene, substituted or unsubstituted arylene, substituted or unsubstituted heteroarylene, or a combination thereof;

R^{N1} is independently hydrogen, substituted or unsubstituted acyl, substituted or unsubstituted, C₁₋₁₀ alkyl, substituted or unsubstituted carbocyclyl, substituted or unsubstituted heterocyclyl, substituted or unsubstituted aryl, substituted or unsubstituted heteroaryl, or an oxygen protecting group;

R^{K2} is halogen, substituted or unsubstituted, C₂₋₁₀ alkenyl, substituted or unsubstituted, C₂₋₁₀ alkynyl, substituted or unsubstituted carbocyclyl, substituted or unsubstituted heterocyclyl, substituted or unsubstituted heteroaryl, -L^{K2}-(substituted or unsubstituted carbocyclyl), -L^{K2}-(substituted or unsubstituted aryl), or —OR^{N2},

L^{K2} is —O—, substituted or unsubstituted, C₁₋₁₀alkylene, substituted or unsubstituted, C₂₋₁₀ heteroalkylene, substituted or unsubstituted carbocyclylene, substituted or unsubstituted heterocyclylene, substituted or unsubstituted arylene, substituted or unsubstituted heteroarylene, or a combination thereof;

R^{N2} is independently hydrogen, substituted or unsubstituted acyl, substituted or unsubstituted, Cr-to alkyl, substituted or unsubstituted carbocyclyl, substituted or unsubstituted heterocyclyl, substituted or unsubstituted aryl, substituted or unsubstituted heteroaryl, or an oxygen protecting group;

or R^{K1} and R² are joined with the intervening atom to form substituted or unsubstituted carbocyclyl or substituted or unsubstituted heterocyclyl;

j is 1, 2, or 3; and
k is 1, 2, or 3;
   provided that the compound is not of the formula:

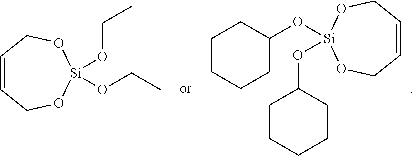 [SMILES: CCO[Si]1(OCC)OCC=CCO1 ; C1=CCO[Si](OC2CCCCC2)(OC2CCCCC2)OC1]

In certain embodiments, the compound is not of the formula:

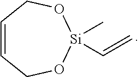

In certain embodiments, the compound comprises one non-aromatic C—C or non-aromatic C≡C bond. In certain embodiments, the compound comprises two, three, or four non-aromatic C═C and/or non-aromatic C═C bonds,:ln certain embodiments, the compound comprises two (i.e., only two) non-aromatic C≡C and/or non-aromatic C≡C bonds, In certain embodiments, the compound comprises only three non-aromatic C═C and/or non-aromatic C≡C bonds (i.e., the combined number of non-aromatic C═C bonds and non-aromatic C≡C bonds is three). In certain embodiments, the compound comprises no CEEC bonds.

In certain embodiments, the compound is of the formula:

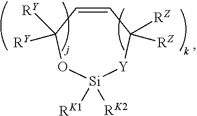

or a salt thereof.

In certain embodiments, the compound is of the formula:

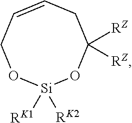

or a salt thereof.

In certain embodiments, the compound is of the formula:

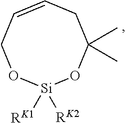

or a salt thereof.

In certain embodiments, the compound is of the formula:

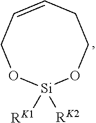

or a salt thereof.

In certain embodiments, the compound is of the formula:

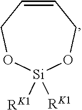

or a salt thereof.

In certain embodiments, the compound is of the formula:

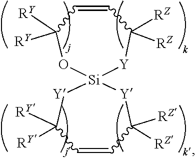

or a salt thereof, wherein:

each instance of Y′ is independently O or C(R^{Q′})₂;

each instance of R^{Q′} is independently hydrogen, halogen, or substituted or unsubstituted, C₁₋₆ alkyl;

each instance of R^{Y′} is independently hydrogen, halogen, or substituted or unsubstituted, C₁₋₆ alkyl;

each instance of R^{Z′} is independently hydrogen, halogen, or substituted or unsubsti ted, C₁₋₆ alkyl;

j′ is 1, 2, or 3; and

k′ is 1, 2, or 3.

The compound of claim 7, or a salt thereof, wherein the compound is of the formula:

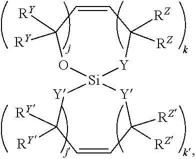

or a salt thereof.

The compound of claim 7, or a salt thereof, wherein the compound is of the formula:

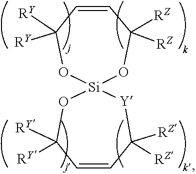

or a salt thereof.

The compound of claim 7, or a salt thereof, wherein the compound is of the formula:

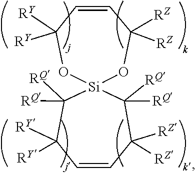

or a salt thereof.

In certain embodiments, each instance of Y′ is O. In certain embodiments, one instance of Y′ is CH₂, and the other instance of Y′ is O.

In certain embodiments, each instance of R^{Q} is hydrogen.

In certain embodiments, each instance of R^{Y′} is hydrogen. In certain embodiments, each instance of R^{Y′} is independently hydrogen or unsubstituted C₁₋₆ alkyl. In certain embodiments, at least one instance of R^{Y′} is substituted or unsubstituted, C₁₋₆ alkyl.

In certain embodiments, each instance of R^{Z′} is hydrogen. In certain embodiments, each instance of is independently hydrogen or unsubstituted C1-6 alkyl. In certain embodiments, at least one instance of R^{Z′} is substituted or unsubstituted, C₁₋₆ alkyl.

In certain embodiments, j′ is 1, and k′ is 1. in certain embodiments, j′ is I, and k′ is 2. In certain embodiments, j′ is 1, and k′ is 3. In certain embodiments, j′ is 2, and k′ is 2. In certain embodiments, j′ is 2, and k′ is 3.

In certain embodiments, W is carbon. in certain embodiments, W is silicon.

In certain embodiments, Y is O. In certain embodiments, Y is CH₂.

In certain embodiments, each instance of R^{Q} is hydrogen.

In certain embodiments, each instance of R^{Y} is hydrogen. In certain embodiments, each instance of R^{Y} is independently hydrogen or unsubstituted C₁₋₆ alkyl. In certain embodiments, at least one instance of R^{Y} is substituted or unsubstituted, C₁₋₆ alkyl.

In certain embZdiments, each instance of R^{Y} is hydrogen. In certain embodiments, each instance of R^{Y} is independently hydrogen or unsubstituted C₁₋₆ alkyl. In certain embodiments, at least one instance of R^{Z}is substituted or unsubstituted, C₁₋₆ alkyl.

In certain embodiments, j is 1, and k is 1. In certain embodiments, j is 1, and k is 2. In certain embodiments, j is 1, and k is 3. In certain embodiments, j is 2, and k is 2. In certain embodiments, j is 2, and k is 3.

In certain embodiments, R^{K1} is substituted or unsubstituted, C₁₋₁₀ alkyl, substituted or unsubstituted, C₂₋₁₀ alkenyl, substituted or unsubstituted, C₂₋₁₀ alkenyl, substituted or unsubstituted carbocyclyl, substituted or unsubstituted heterocyclyl, substituted or unsubstituted aryl, substituted or unsubstituted heteroaryl, -L^{K1}-(substituted or unsubstituted carbocyclyl), -L^{K1}-(substituted or unsubstituted heterocyclyl), -L^{K1}-(substituted or unsubstituted aryl), -L^{K1}-(substituted or unsubstituted heteroaryll), or —OR^{N1}.

In certain embodiments, R^{K1} is substituted or unsubstituted, C₁₋₁₀ alkyl. In certain embodiments, R^{K1} is unsubstituted methyl, unsubstituted ethyl, unsubstituted propyl (e.g., unsubstituted n-propyl or unsubstituted isopropyl), or unsubstituted butyl (e.g., unsubstituted butyl).

In certain embodiments, R^{K1} is substituted or unsubstituted, saturated carbocyclyl. In certain embodiments, R^{K1} is unsubstituted cyclopropyl, unsubstituted cyclobutyl, unsubstituted cyclopentyl, unsubstituted cyclohexyl, or unsubstituted cycloheptyl.

In certain embodiments, R^{K1} is substituted or unsubstituted, partially unsaturated carbocyclyl, in certain embodiments, R^{K1} is substituted or unsubstituted carbocyclyl that comprises only one unsaturated bond in the carbocyclic ring system. In certain embodiments, R^{K1} is unsubstituted cyclobutenyl, unsubstituted cyclopentenyl, unsubstituted cyclohexenyl, or unsubstituted cycloheptenyl. In certain embodiments, R^{K1} is substituted or unsubstituted carbocyclyl that comprises only two unsaturated bonds in the carbocyclic ring system. In certain embodiments, R^{K1} is substituted or unsubstituted carbocyclyl that comprises no C≡bonds in the carbocyclic ring system.

In certain embodiments, R^{K1} is:

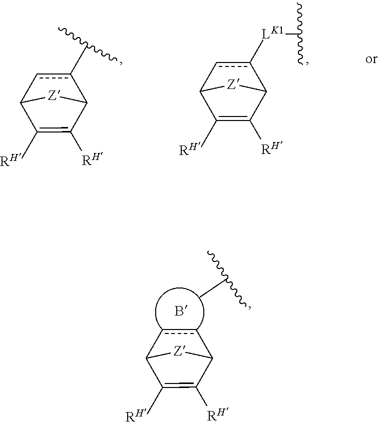

wherein:

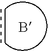

is Ring B′, wherein Ring B′ is a substituted or unsubstituted, monocyclic carbocyclic ring, substituted or unsubstituted, monocyclic heterocyclic ring, substituted or unsubstituted, monocyclic aryl ring, or substituted or unsubstituted, monocyclic heteroaryl ring;

Z′ is C(R^{P′})₂ or O;

each instance of R^{P′} is independently hydrogen, halogen, or substituted or unsubstituted, C₁₋₆ alkyl;

[symbol]

is a single bond or double bond; each instance of R^{H′} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —OCN, —OC(═O)R^{a}, —OC)═S)R^{a}, —OC(═O)OR⁴, OC(═O)N(R^{a})₂, —OS(═O)R¹, —OS(═O)R³, —OS(═O)OR^{a}, —OS(═O)N(R^{a})₂, —OS(═O)₂R^{a}, —OS(═O)₂OR^{a}, —OS(═O)₂N(R³)₂, —OSi((R^{a}). —OSi(R^{a})₂(OR^{a}), —OSi(R³)(OR³)₂, —OSi(OR³)₃, oxo, —N(R^{a})₃, —N═C(R^{a})₂, ═NR^{a}, —NC, —NCO, —N₃, —NO₂, —NR^{a}C(═O)R^{a}, —NR^{a}C(═O)OR³, —NR^{a}C(═O)N(R^{a}₂, —NR^{a}S(═O)R^{a}, —NR^{a}S(═O)OR^{a}, —NR^{a}S(═O)OR^{a}, —NR^{a}S(═O)N(R^{a})₂, —NR^{a}NR³S(═O)₂R^{a}, —NR^{a}S(═O)₂OR^{a}, —NR^{a}S(═O)₂N(R^{a})₂, —SR^{a}, —SCN, —S(═O)R^{a}, —S(═O)OR^{a}, —S(═O)N(R³)₂, —S(═O)₂R^{a}, —S(═O)₂OR^{a}, —S(═O)₂N(R^{a}₂; —SeR^{a}, —SeR^{a}, halogen, —CN, —C(═NR^{a}(R^{a}, —C(═NR^{a})OR^{a}, —C(═NR^{a})OR^{a}, —C(═NR^{a})N(R^{a}), —C(═O)R^{a}), —C(═O)OR^{a}, C(═O)SR^{a}, −C(═S)OR^{a}, or —C(═O)N(R^{a})₂;

or the two instances of R^{H′} are ioined with the intervening carbon atoms to form a substituted or unsubstituted, monocyclic carbocyclic ring, substituted or unsubstituted, monocyclic heterocyclic ring, substituted or unsubstituted, monocyclic aryl ring, or substituted or unsubstituted, monocyclic heteroaryl ring; and

each instance of R^{a} is independently hydrogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted, monocyclic aryl, substituted or unsubstituted, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form substituted or unsubstituted heterocyclyl or substituted or unsubstituted heteroaryl.

In certain embodiments, R^{K1} is:

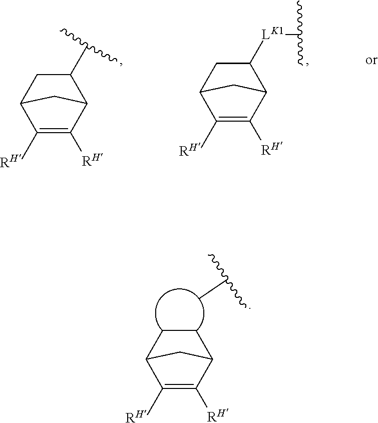

In certain embodiments, Ring B′ is substituted or unsubstituted, monocyclic carbocyclic ring. In certain embodiments, Ring B′ is substituted or unsubstituted, monocyclic, saturated carbocyclyl. In certain embodiments, Ring B′ is unsubstituted cyclopropyl, unsubstituted cyclobutyl, unsubstituted cyclopentyl, unsubstituted cyclohexyl, or unsubstituted cycloheptyl. In certain embodiments, Ring B′ is substituted or - unsubstituted, monocyclic, partially unsaturated carbocyclyl. In certain embodiments, Ring B′ is substituted or unsubstituted, monocyclic carbocyclyl that comprises only one unsaturated bond in the carbocyclic ring system. In certain embodiments, Ring B′ is unsubstituted cyclobutenyl, unsubstituted cyclopentenyl, unsubstituted cyclohexenyl, or unsubstituted cycloheptenyl. in certain embodiments, Ring B′ is substituted or unsubstituted, monocyclic carbocyclyl that comprises only two unsaturated bonds in the carbocyclic ring system. in certain embodiments, Ring B′ is substituted or unsubstituted, monocyclic carbocyclyl that comprises no C≡C bonds in the carbocyclic ring system.

In certain embodiments, Z′ is CH₂.

In certain embodiments, each R^{H′} is hydrogen. In certain embodiments, two instances of R^{H′} are joined with the intervening carbon atoms to form an unsubstituted monocyclic carbocyclic ring. In certain embodiments, two instances R^{H′} are joined with the intervening carbon atoms to form an unsubstituted monocylic heterocyclic ring.

In certain embodiments, R^{K1} is

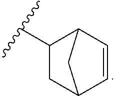 [SMILES: *C1CC2C=CC1C2]

In certain embodiments, R^{K1} is -(substituted or unsubstituted, C₁₋₁₀ alkylene)-(substituted or unsubstituted, partially saturated carbocyclyl). In certain embodiments, R^{K1} is -(unsubstituted C140 alkylene)-(substituted or unsubstituted carbocyclyl that comprises only one unsaturated bond in the carbocyclic ring system). In certain embodiments, R^{H′} is substituted or unsubstituted heterocyclyl or L^{K1}-(substituted or unsubstituted heterocyclyl). In certain embodiments, R^{K1} is substituted or unsubstituted heterocyclyl that comprises O—Si in the heterocyclic ring system or -L^{K1}-(substituted or unsubstituted heterocyclyl that comprises O—Si in the heterocyclic ring system). In certain embodiments, R^{K1} is -(substituted or unsubstituted, C₁₋₁₀ alkylene)-(substituted ter unsubstituted heterocyclyl). In certain embodiments, R^{K1} is —(substituted or unsubstituted, C₁₋₁₀ alkylene)-substituted or unsubstituted heterocyclyl that comprises O—Si in the heterocyclic ring system). In certain embodiments, R^{K1} is:

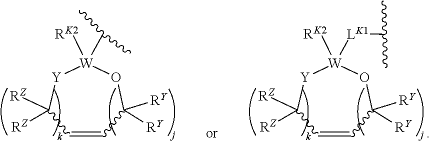

In certain embodiments, R^{K1} is -(substituted or unsubstituted phenylene)-(substituted or unsubstituted, partially saturated heterocyclyl). In certain embodiments, R^{K1} is -(substituted or unsubstituted phenylene)-(substituted or unsubstituted heterocyclyl that comprises only one unsaturated bond in the heterocyclic ring system). In certain embodiments, R^{K1} is -(substituted or unsubstituted phenylene)-(substituted or unsubstituted, partially saturated heterocyclyl that comprises O═Si in the heterocyclic ring system). In certain embodiments, R^{K1} is -(substituted or unsubstituted phenylene)-(substituted or unsubstituted heterocyclyl that comprises O—Si and only one unsaturated bond in the heterocyclic ring system).

In certain embodiments, R^{K1} is

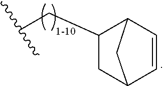

In certain embodiments, R^{K1} is

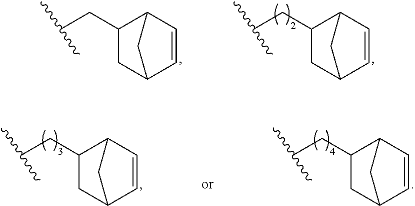

In certain embodiments, R^{K1} is

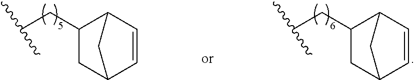

In certain embodiments, R^{K1} is hydrogen. In certain embodiments, R^{K1} is —OR^{N1}.

In certain embodiments, R^{N1} is substitued or unsubstituted, C₁₋₁₀ alkyl. In certain embodiments, R^{N1} is unsubstituted C₁₋₂ alkyl.

In certain embodiments, R^{K2} is substituted or unsubstituted, C₁₋₁₀ alkylene. In certain embodiments, L^{K1} is unsubstituted. C₁₋₁₀ alkylene. In certain embodiments, L^{K3} is substituted or unsubstituted phenylene. In certain embodiments, R^{K2} is substituted or unsubstituted, saturated carbocyclyl. In certain embodiments, R^{K2} is unsubstituted cyclopropyl, unsubstituted cyclobutyl, unsubstituted cyclopentyl, unsubstituted cyclohexyl, or unsubstituted cycloheptyl.

In certain embodiments, R^{K2} is substituted or unsubstituted, partially unsaturated carbocyclyl. In certain embodiments, R^{K2} is substituted or unsubstituted carbocyclyl that commises only one unsaturated bond in the carbocyclic ring system. In certain embodiments, R^{K2} is unsubstituted cyclobutenyl, unsubstituted cyclopentenyl, unsubstituted cyclobexenyl, or unsubstituted cycloheptenyl. In certain embodiments, R^{K2} is substituted or unsubstituted carbocyclyl that comprises only two unsaturated bonds in the carbocyclic ring system. In certain embodiments, R^{K1} is substituted or unsubstituted carbocyclyl that comprises no C≡C bonds in the carbocyclic ring system.

in certain embodiments, R^{K2} is:

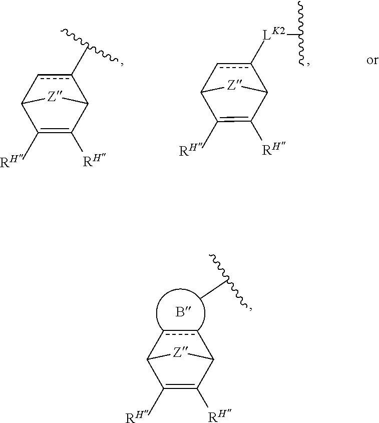

wherein:

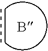

is Ring B″, wherein Ring B″ is a substituted or unsubstituted, monocyclic carbocyclic ring, substituted or unsubstituted, monocyclic heterocyclic ring, substituted or unsubstituted, monocyclic aryl ring, or substituted or unsubstituted, tnonocydic heteroaryl ring;

Z″ is C(R^{P″)}₂ or O;

each instance of R^{P″} is independently hydrogen, halogen, or substituted or unsubstituted, C₁₋₆ alkyl;

[symbol] is a single bond or double bond;

each instance of R^{H″} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, —OR^{a}, —OCN, —OC(═O)R^{a}, —OC(═S)R^{a}, —OC(═O)OR^{a}, —OC(═O)N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, —OSi(R^{a})₂, —OSi(R^{a})₂(OR^{a}), —OSi(R^{a})(OR^{a})₂, —OSi(OR^{a})(OR^{a})₂, —OSi(OR^{a})₃, oxo, —N(R^{a})₂, —N═C(R^{a})₂, ═NR^{a}, —NC, —NCO, —N₃, —NO₂, —NR^{a}C(═O)R^{a}, —NR^{a}C(═O)(OR^{a}, —NR^{a}C(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂, N(R^{a})₂, —SR^{a}, —SCN, —S(═O)R^{a}, —S(═O)OR^{a}, —S(═O)N(R^{a}, —C(αNR^{a})N(R^{a}), —C(═O)R^{a}, —C═O(R^{a}, —C(═O)OR^{a}, —C(═O)SR^{a}, —C(═S)OR^{a}, or —C(═O)N(R^{a})₂;

or the two instances of R^{H+} are kpomed wotj tje omtervemomg carbpm atp,s tp fpr, a substituted or unsubstituted, monocyclic carbocyclic ring, substituted or unsubstituted, monocyclic heterocyclic ring, substituted or unsubstituted, monocyclic aryl ring, or substituted or unsubstituted, monocyclic heteroaryl ring; and

each instance of R^{a} is independently hydrogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted, monocyclic aryl, substituted or unsubstituted, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form substituted or unsubstituted heterocyclyl or substituted or unsubstituted heteroaryl,

In certain embodiments, R^{K2} is:

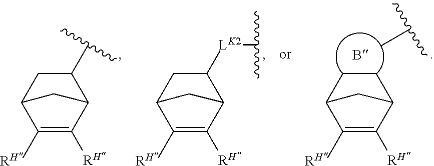

In certain embodiments, :Ring B″ is substituted or unsubstituted, monocyclic carbocyclic ring. In certain embodiments, Ring B″ is substituted or unsubstituted, monocyclic, saturated carbocyclyl. In certain embodiments, Ring B″ is unsubstituted cyclopropyl, unsubstituted cyclobutyl, unsubstituted. cyclopentyl, unsubstituted cyclohexyl, or unsubstituted cycloheptyl. In certain embodiments, Ring B″ is substituted or unsubstituted, monocyclic, partially unsaturated carbocyclyl. In certain embodiments, Ring B″ is substituted or unsubstituted, monocyclic carbocyclyl that comprises only one unsaturated bond in the carbocyclic ring system. In certain embodiments, Ring B″ is unsubstituted unsubstituted cyclopentenyl, unsubstituted cyclohexenyl, or unsubstituted cycloheptenyl. In certain embodiments, Ring B″ is substituted or unsubstituted, monocyclic carbocyclyl that comprises only two unsaturated bonds in the carbocyclic ring system. In certain embodiments, Ring B″ is substituted or unsubstituted, monocyclic carbocyclyl that comprises no C≡C bonds in the carbocyclic ring system.

In certain embodiments, Z″ is CH₂.

In certain embodiments, each R^{H″} is hydrogen. In certain embodiments, two instances of R^{H″} are joined with the intervening carbon atoms to form an unsubstituted monocyclic carbocyclic ring. In certain embodiments, two instances of R^{H″} are joined with the intervening carbon atoms to form an unsubstituted monocylic heterocyclic ring.

In certain embodiments, R^{K2} is

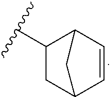 [SMILES: *C1CC2C=CC1C2]

In certain embodiments, R^{K2} is —(substituted or unsubstituted, C₁₋₁₀ alkylene)-(substituted or unsubstituted, partially saturated carbocyclyl). In certain embodiments, R^{K2} is -(unsubstituted C140 alkylene)—(substituted or unsubstituted carbocyclyl that comprises only one unsaturated bond in the carbocyclic ring system).

In certain embodiments, R^{K2} is substituted or unsubstituted heterocyclyl or (substituted or unsubstituted heterocyclyl). In certain embodiments, R^{K2} is substituted or unsubstituted heterocyclyl that comprises O—Si in the heterocyclic ring system or -L^{K2}-(substituted or unsubstituted heterocyclyl that comprises O—Si in the heterocyclic ring system). In certain embodiments, R^{K2} is -(substituted or unsubstituted, C₁₋₁₀ alkylene)-(substituted or unsubstituted heterocyclyl). In certain embodiments, R^{K2} -(substituted or unsubstituted, C₁₋₁₀ alkylene)--substituted or unsubstituted heterocyclyl that comprises O═Si in the heterocyclic ring system). In certain embodiments, R^{K2} is:

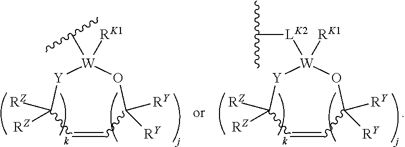

In certain embodiments, R^{K2} is -(substituted or unsubstituted phenylene)-(substituted or unsubstituted, partially saturated heterocyclyl). In certain embodiments, R^{K2} is -(substituted or unsubstituted phenylene)-(substituted or unsubstituted heterocyclyl that comprises only one unsaturated bond in the heterocyclic ring system). In certain embodiments, R^{K2} is -(substituted or unsubstituted phenylene)-(substituted or unsubstituted, partially saturated heterocyclyl that comprises O—Si in the heterocyclic ring system). In certain embodiments, R^{K2} is -(substituted or unsubstituted phenylene)-(substituted or unsubstituted heterocyclyl that comprises O—Si and only one unsaturated bond in the heterocyclic ring system).

In certain embodiments, R^{K2} is

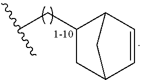

In certain embodiments, R^{K2} is

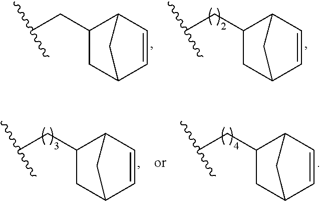

In certain embodiments, R^{K2} is

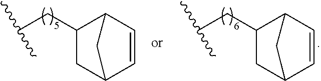

In certain embodiments, R^{K2} is hydrogen. in certain embodiments, R^{K2} is —OR^{N2}.

In certain embodiments, R^{N2} is substituted or unsubstituted, C₁₋₁₀ alkyl. In certain embodiments, R^{N2} is unsubstituted C₁₋₆ alkyl.

In certain embodiments, L^{K2} is —O— or substituted or unsubstituted, C₁₋₁₀ alkylene. In certain embodiments, L^{K2} is unsubstituted C₁₋₆ alkylene. In certain embodiments, L^{K2} is substituted or unsubstituted phenylene.

In certain embodiments, R^{K1} and R^{K2} are joined with the intervening atom to form substituted or unsubstituted, partially unsaturated carbocyclyl. In certain embodiments, R^{K1} and R^{K2} are joined with the intervening atom to form substituted or unsubstituted, monocyclic carbocyclyl that comprises only one unsaturated bond in the carbocyclic ring system. In certain embodiments, R^{K1} and R^{K2} are joined with the intervening atom to form unsubstituted cyclobutenyl, unsubstituted cyclopentenyl, unsubstituted cyclohexenyl, or unsubstituted cycloheptenyl. In certain embodiments, R^{K1} and R^{K2} are joined with the intervening atom to form substituted or unsubstituted, monocyclic carbocyclyl that comprises only two unsaturated bonds in the carbocyclic ring system. In certain embodiments, R^{K1}and R^{K2} are joined with the intervening atom to form substituted or unsubstituted carbocyclyl that comprises no C═C bonds in the carbocyclic ring system.

In certain embodiments, R^{K1} and R^{K2} are joined with the intervening atom to form substituted or unsubstituted, partially unsaturated heterocyclyi. In certain embodiments, R^{K1} and R^{K2} are joined with the intervening atom to form substituted or unsubstituted, monocyclic heterocyclyl that comprises only one unsaturated bond in the heterocyclic ring system. In certain embodiments, R^{K1} and R^{K2} are joined with the intervening atom to form substituted or unsubstituted, monocyclic heterocyclyl that comprises only two unsaturated bonds in the heterocyclic ring system. In certain embodiments, R^{K1} and R^{K2} are joined with the intervening atom to form substituted or unsubstituted heterocyclyl that comprises no C═C bonds in the heterocyclic ring system.

In certain embodiments, the C═C bond in the heterocyclic ring that comprises O—W—Y is of the (Z)-configuration. In certain embodiments, the C═C bond in the heterocyclic ring that comprises O—W—Y is of the (F)-configuration.

In certain embodiments, the compound is of the formula:

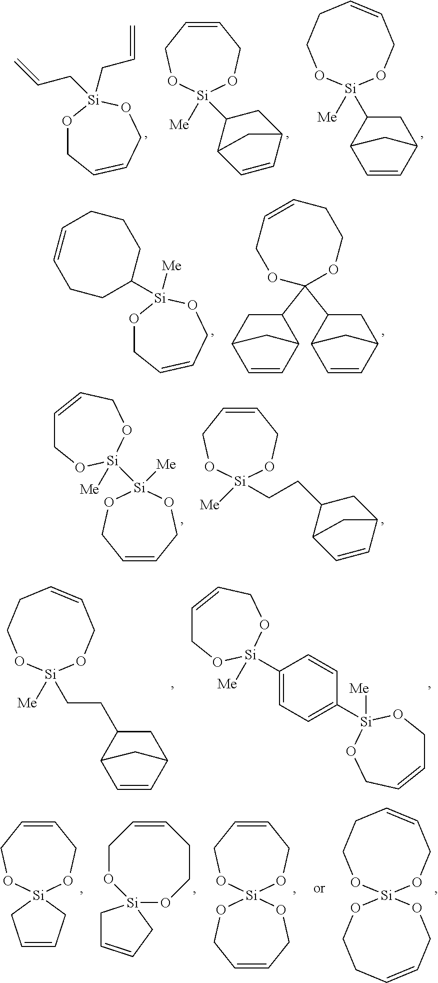

or a salt thereof.

In certain embodiments, the compound is of the formula:

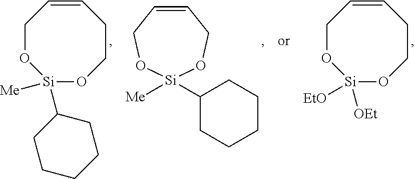

or a salt thereof.

In certain embodiments, the compound is of the formula:

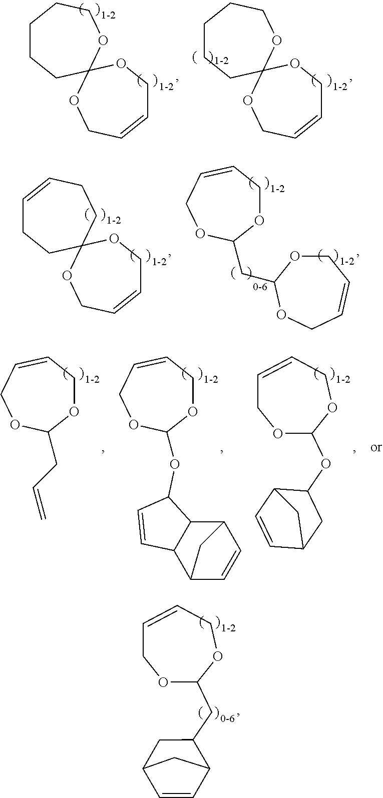

or a salt thereof.

In certain embodiments, a compound of the present disclosure is a compound of Formula (B), or a salt thereof.

In another aspect, the present disclosure provides copolymers (copolymers of the present disclosure) prepared by a method comprising polymerizing:

one or more instances of a first monomer;

one or more instances of a second monomer, wherein the second monomer is a compound of the present disclosure, or a salt thereof; and

optionally one or more instances of a third monomer;

wherein any two instances of the first monomer are the same as or different from each other, any two instances of the second monomer are the same as or different from each other, any two instances of the third monomer are the same as or different from each other, and each instance of the first monomer, the second monomer, and the third monomer if present, is different from each other;

in the presence of a metathesis catalyst.

In another aspect, the present disclosure provides methods of preparing a copolymer of the present disclosure comprising polymerizing:

one or more instances of a first monomer; one or more instances of a second monomer, whereine second monomer is a compound of the present disclosure, or a salt thereof; and

optionally one or more instances of a third monomer;

wherein any two instances of the first monomer are the same as or different from each other, any two instances of the second monomer are the same as or different from each other, any two instances of the third monomer are the same as or different from each other, and each instance of the first monomer, the second monomer, and the third monomer if present, is different from each other;

in the presence of a metathesis catalyst.

In another aspect, the present disclosure provides hydroxylated oligomers (hydroxylated oligomers of the present disclosure) or hydroxylated polymers (hydroxylated polymers of the present disclosure) prepared by a method comprising hydrolyzing a copolymer of the present disclosure, wherein the step of hydrolyzing the copolymer comprises hydrolyzing one or more instances of the —O—Si bonds of the copolymer to form —OH.

In another aspect, the present disclosure provides methods of preparing a hydroxylated oligomer or hydroxylated polymer comprising hydrolyzing a copolymer of the present disclosure, wherein the step of hydrolyzing the copolymer comprises hydrolyzing one or more instances of the —O—Si bonds of the copolymer to form —OH.

In certain embodiments, the step of polymerizing (Step (a)) is substantially free of solvents. In certain embodiments, Step (a) further comprises the presence of a solvent (e.g., organic solvent).

In certain embodiments, Step (a) is substantially free of a chain transfer agent.

In certain embodiments, the temperature of Step (a) is between 20 and 40, between 40 and 60, between 60 and 80, between 80 and 100, between 100 and 120, between 120 and 140, or between 140 and 160° C., inclusive, In certain embodiments, the temperature of Step (a) is between 100 and 140° C., inclusive.

In certain embodiments, the time duration of Step (a) is between 1 and 10 minutes, between 10 and 60 minutes, between 1 and 6 hours, between 6 and 24 hours, between 1 and 3 days, or between 3 and 7 days, inclusive. In certain embodiments, the time duration of Step (a) is between 10 minutes and 2 hours, inclusive.

In certain embodiments, the polymerizing of Step (a) is ROMP.

In certain embodiments, the method of preparing the copolymer further comprises (b) exposing the copolymer to a solvent.

In certain embodiments, the method of preparing the copolymer further comprises (c) solid-liquid phase separation. In certain embodiments, Step (c) is subsequent to Step (b).

In certain embodiments, the method of preparing the copolymer further comprises curing. In some embodiments, curing forms a resin. In certain embodiments, curing is carried out at 70 to 150° C., inclusive. In certain embodiments, curing is carried out at 100 to 150 ° C., inclusive. In certain embodiments, curing is carried out at 100 to 130° C., inclusive. In certain embodiments, curing is carried out at 110 to 120° C., inclusive. In some embodiments, curing is carried out at about 110° C. in some embodiments, curing is carried out at about 120° C. In some embodiments, curing is carried out for 1 minute to 3 hours, inclusive. In some embodiments, curing is carried out for 15 minutes to 1 hour, inclusive. In some embodiments, curing is carried out for 15 minutes. In certain embodiments, curing is carried out for 30 minutes. In some embodiments, curing is carried out for 1 hour. In certain embodiments, curing is carried out at ambient pressure. In some embodiments, curing is carried out at lower-than-ambient pressure. In some embodiments, curing is carried out at higher-than-ambient pressure.

The preparation of the copolymers may involve a metathesis reaction. In certain embodiments, the metathesis reaction is a ring-opening metathesis copolymerization (ROMP) (see, e.g., Liu el at Am. Chem. Soc. 2012, 134, 16337; Liu, J.; Gao, A. X.; Johnson, J. A. J Vis Exp 2013, e50874).

In certain embodiments, the metathesis catalyst (e.g., ROMP catalyst) is a tungsten (W), molybdenum (Mo), or ruthenium (Ru), metathesis catalyst. In certain embodiments, the metathesis catalyst is a ruthenium metathesis catalyst. Metathesis catalysts useful in the synthetic methods described herein include catalysts as depicted below, and as described in Grubbs et al., Acc. Chem, Res. 1995, 28, 446-452; U.S. Pat. No. 5,811,515; Schrock et al., Organometallics (1982) 1 1645; Gallivan et al., Tetrahedron Leiters (2005) 46:2577-2580; Furstner et al., J. Am. Chem, Soc. (1999) 121:9453; and Chem. Eur. J. (2001) 7:5299; the entire contents of each of which are incorporated herein by reference.

In certain embodiments, the metathesis catalyst is a Grubbs catalyst. In certain embodiments, the Grubbs catalyst is selected from the group consisting of:

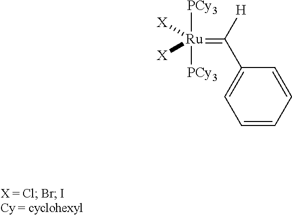

Benzylidenebis-(tricyclohexylphosphine)-dichlororutheniurn (X═Cl); Benzyliclenebis-(tricyclohexylphosphine)-dibromoruthenium (X═Br); Benzylidenebis-(tricyclohexylphosphine)-diiodonithenium (X═I);

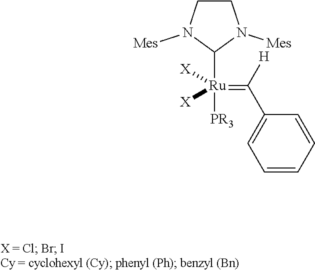

1,3-(Bis(mesityl)-2-imidazolidinylidene)dichloro-(phenyhnethylene) (tricyclohexyl-phosphine)ruthenium (X ═Cl; R=cyclohexyl); 1,3-(Bis(mesityl)-2-imidazolidinylidene)dibromo-(phenyhnethylene) (tricyclohexyl-phosphine)nithertium (X═Br; R=cyclohexyl); 1,3-(Bis(mesityl)-2-imidazolidinylidene)diiodo-(phenylmothylene) (tricyclohexyl-phosphine)rathenium (X═I; R=cyclohexyl); 1,3-(Bis(mesityl)-2-imidiazolidinylidene)dichloro-(phenylmothylene) (triphenylphosphine)ruthenium (X═Cl; R=phenyl); 1,3-(Bis(mesityl)-2-imidazolidinylidene)dichloro-(phenylmethylene) (tribenzylphosphine)ruthenium (X═Cl; R=benzyl);

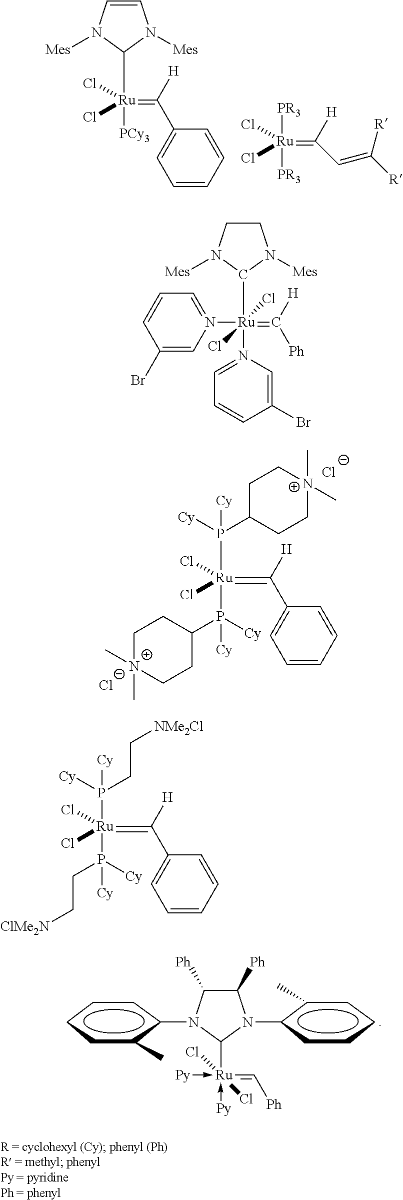

In certain embodiments, the metathesis catalyst is a Grubbs-Hoveyda catalyst. In certain embodiments, the Grubbs-Hoveyda catalyst is selected from the group consisting of:

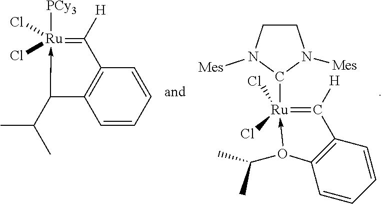

In certain embodiments, t e metathesis catalyst is selected from the group consisting of:

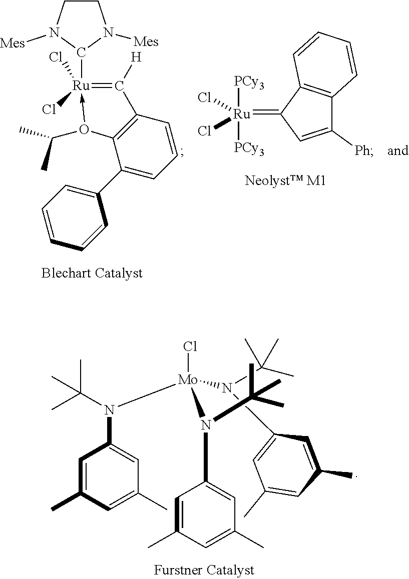

In certain embodiments, the metathesis catalyst is of the formula:

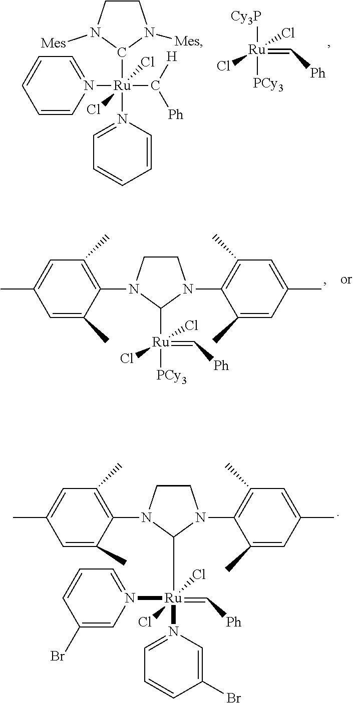

In certain embodiments, the metathesis catalyst is the second-generation Grubbs catalyst. In certain embodiments, the ratio of the combined molar amounts of the first monomer, second monomer, and third monomer if present to the molar amount of the metathesis catalyst is not less than 1,000. In certain embodiments, the ratio of the combined molar amounts of the first monomer, second monomer, and third monomer if present to the molar amount of the metathesis catalyst is between 100 and 300, inclusive. In certain embodiments, the ratio of the combined molar amounts of the first monomer, second monomer, and third monomer if present to the molar amount of the metathesis catalyst is between 300 and 1,000, inclusive. in certain embodiments, the ratio of the combined molar amounts of the first monomer, second monomer, and third monomer if present to the molar amount of the metathesis catalyst is between 1,000 and 1,50©, inclusive. In certain embodiments, the ratio of the combined molar amounts of the first monomer, second monomer, and third monomer if present to the molar amount of the metathesis catalyst is between 1,500 and 2,000, inclusive. In certain embodiments, the ratio of the combined molar amounts of the first monomer, second monomer, and third monomer if present to the molar amount of the metathesis catalyst is between 2,000 and 10,000, inclusive. In certain embodiments, the ratio of the combined molar amounts of the first monomer, second monomer. and third monomer if present to the molar amount of the metathesis catalyst is between 10,000 and 30,000, inclusive. In certain embodiments, the ratio of the combined molar amounts of the first monomer, second monomer, and third monomer if present to the molar amount of the metathesis catalyst is between 30,000 and 100,000, inclusive.

The ROMP can he conducted in one or more aprotic solvents. The term “aprotic solvent” means a non-nucleophilic solvent having a boiling point range above ambient temperature, preferably from about 25° C. to about 190° C. at atmospheric pressure. In certain embodiments, the aprotic solvent has a boiling point from about 80° C. to about 160° C. at atmospheric pressure. In certain embodiments, the aprotic solvent has a boiling point from about 80° C. to about 150° C. at atmospheric pressure. Examples of such solvents are methylene chloride, acetonitrile, toluene, DMF, diglyme, THF, and DMSO.

The ROMP can be quenched with a vinyl ether of the formula

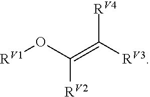

Each of R^{V1}, R^{V2}, R^{V3}, and R^{V4} is independently optionally substituted alkyl, optionally substituted alkenyl, optionally substituted alkynyl, optionally substituted carbocyclyl, optionally substituted phenyl, optionally substituted heterocyclyl, or optionally substituted heteroaryl, In certain embodiments, R^{V1} is optionally substituted alkyl, and R^{V2}, R^{V3}, and R^{V4} are hydrogen. In certain embodiments, R^{V1} is unsubstituted alkyl, and R^{V2}, R^{V3}, and R^{V4} are hydrogen. In certain embodiments, R^{V1} is substituted alkyl, and R^{V2}, R^{V3}, and R^{V4} are hydrogen. In certain embodiments, R^{V1} is methyl, and R^{V2}, R^{V3}, and R^{V4} are hydrogen. In certain embodiments, R^{V1} is ethyl, and R^{V2}, R^{V3}, and R^{V4} are hydrogen. In certain embodiments, R^{V1} is propyl, and R^{V2}, R^{V3}, and R^{V4} are hydrogen. In certain embodiments, R^{V1} is optionally substituted alkenyl, and R^{V2}, R^{V3}, and R^{V4} are hydrogen. In certain embodiments, R^{V1} is unsubstituted alkenyl, and R^{V2}, R^{V3}, and R^{V4} are hydrogen. In certain embodiments, R^{V1} is vinyl, and R^{V2} and R^{V3}, and R^{V4} are hydrogen. In certain embodiments, at least one of R^{V1}, R^{V2}, R^{V3}, and R^{V4} is conjugated with a diagnostic agent as defined above. in certain embodiments, the ROMP is quenched by ethyl vinyl ether. Excess ethyl vinyl ether can be removed from the copolymer under reduced pressure.

In certain embodiments, at least two instances of a variable (e.g., a moiety) are different from each other. In certain embodiments, all instances of a variable are different from each other. In certain embodiments, all instances of a variable are the same. For example, when a compound, copolymer, hydroxylated oligomer, or hydroxylated polymer comprises two or more instances of a moiety, any two instances of the moiety may be the same or different from each other, unless otherwise provided. For example, when a compound of Formula (B) comprises two instances of R^{K1}, the two instances of R^{K1} may be the same or different from each other.

In certain embodiments, at least one instance of the first monomer is of Formula:

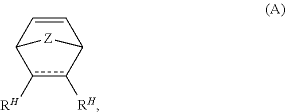

or salt thereof, wherein

each instance of Z is independently C(R^{P})₂ or O;

each instance of R^{P} is independently hydrogen, halogen, or substituted or unsubstituted, C₁₋₁₀ alkyl;

each instance of [symbol] is independently a single bond or double bond;

each instance of R^{H} is independently hydrogen, halogen, substituted or unsubstituted alkyl, substituted or unsubstituted alkenyl, substituted or unsubstituted alkynyl, substituted or unsubstituted carbocyclyl, substituted or unsubstituted heterocyclyi, substituted or unsubstituted aryl, substituted or unsubstituted heteroaryl, —OR^{a}, —OCN, —OC(═O)R^{a}, —OC(═S)R^{a}, —OC(═O)OR³, —OC(═O)N(R³)₂, —OS(═O)R^{a}, —OS(═O)OR⁴, —OC(═O)N(R^{a})₂, —OS(═O)₂R^{a}, —OS(═O)₂OR^{a}, —OS(═O₂N(R^{a})₂, —OS(R^{a}), —OS(R^{a})₂(OR^{a}), —OS(R^{a}((OR^{a})₂, —OSi(OR^{a})₃), oxo, —N(R^{a})₂, —N═C(R^{a})₂, ═NR^{a}, —NC, —NCO, —N₃, —NO₂, —NR^{a}C(═O)R^{a}, —NR^{a}C(═O)OR^{a}, —NR^{a}C(═O)N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —SR^{a}, —S(═O)R^{a}, —S(═O)OR^{a}, —S(═O)N(R^{a})₂, —S(═O)₂R^{a}, —S(═O)₂OR^{a}, —S(═O)₂N(R^{a})₂, —SeR^{a}, halogen, —CN, —C(═NR^{a})R^{a}, —C(═NR^{a})OR^{a}, —C(═NR^{a})N(R^{a})₂, —C(═NR^{a})N(R^{a})₂, —C(═O)R^{a}, —C(═O)OR^{a}, —C(═O)SR², —C(═S)OR², or —C(═O)N(R³)₂,

or the two instances of R^{H} of one or more instances of

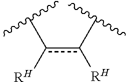

are joined with the intervening carbon atoms to independently form a substituted or unsubstituted, monocyclic carbocyclic ring, substituted or unsubstituted, monocyclic heterocyclic ring, substituted or unsubstituted, monocyclic aryl ring, or substituted or unsubstituted, monocyclic heteroaryl ring; and

each instance of R^{a} is independently hydrogen, substituted or unsubstituted alkyl, substituted or unsubstituted alkenyl, substituted or unsubstituted alkynyl, substituted or unsubstituted, monocyclic carbocyclyl, substituted or unsubstituted, monocyclic heterocyclyl, substituted or unsubstituted, monocyclic aryl, substituted or unsubstituted, monocyclic heteroaryl, a nitrogen protecting group when attached to a nitrogen atom, an oxygen protecting group when attached to an oxygen atom, or a sulfur protecting group when attached to a sulfur atom, or two instances of R^{a} are joined to form substituted or unsubstituted heterocyclyl or substituted or unsubstituted heteroaryl.

In certain embodiments, each instance of the first monomer is independently of Formula (D1) or (D2):

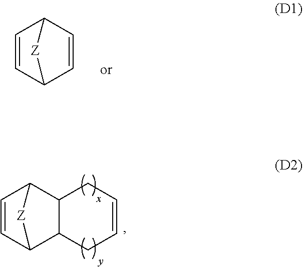

or a salt thereof, wherein:

each instance of x is independently 0, 1, or 2; and

each instance of y is independently 0, 1, or 2.

In certain embodiments, at least one instance of Z is C(R^{P})₂. In certain embodiments, each instance of Z is C(R^{P})₂. In certain embodiments, at least one instance of Z is CH₂. In certain embodiments, each instance of Z is CH₂.

In certain embodiments, each instance of R^{P} is hydrogen. In certain embodiments, at least one instance of R^{P} is hydrogen. In certain embodiments, at least one instance of R^{P} is halogen. In certain embodiments, at least one instance of R^{P} is unsubstituted, C₁₋₆ alkyl or C₁₋₆ alkyl substituted with one or more halogen. In certain embodiments, at least one instance of R^{P} is unsubstituted methyl.

In certain embodiments, at least one instance of R^{H} is hydrogen. In certain embodiments, each instance of R^{H} is hydrogen.

In certain embodiments, at least one instance of R^{H} is substituted or unsubstituted alkyl (e.g., —CF₃). In certain embodiments, at least one instance of R^{H} is —CN. In certain embodiments, at least one instance of R^{H} is —C(═O)OR^{a} (e.g., —C(═P)OCH₃). In certain embodiments, at least one instance of R^{H} is —C(═O)R^{a}. In certain embodiments, at least one instance of R^{H} is —C(═O)N(R^{a}).

In certain embodiments, each instance of the linear units is of the formula:

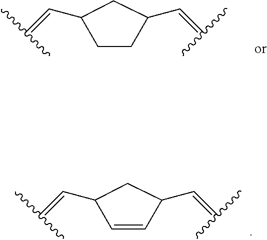 [SMILES: *=CC1CCC(C=*)C1 ; *=CC1C=CC(C=*)C1]

In certain embodiments, each instance of the first monomer is of Formula (D1), or a salt thereof. In certain embodiments, each instance of the first monomer is of Formula (D1).

In certain embodiments, at least one instance of the first monomer is of the formula:

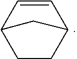

In certain embodiments, each instance of the first monomer is of the formula:

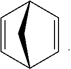

In certain embodiments, each instance of the first monomer is of the formula:

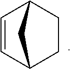

In certain embodiments, the two instances of R^{H} of one or more instances of

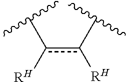

are joined with the intervening carbon atoms to independently form a substituted or unsubstituted, monocyclic carbocyclic ring, or substituted or unsubstituted, monocyclic heterocyclic ring. In certain embodiments, the two instances of R^{H} of one or more instances of

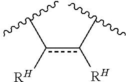

are joined with the intervening carbon atoms to independently form a substituted or unsubstituted, monocyclic cycloalkenyl ring. In certain embodiments, the two instances of R^{H} of one or more instances of

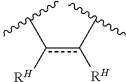

are joined with the intervening carbon atoms to independently form a substituted or un substituted, monocyclic, saturated heterocyclic ring. In certain embodiments, at least one instance of the first monomer comprises a substituted or unsubstituted partially unsaturated monocyclic carbocyclic ring or a substituted or unsubstituted partially unsaturated monocyclic heterocyclic ring.

certain embodiments, each instance of the linear units is of the formula:

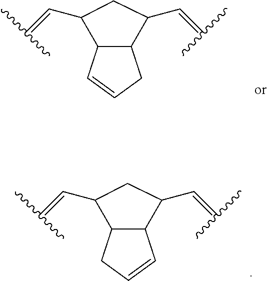 [SMILES: *=CC1CC(C=*)C2CC=CC12 ; *=CC1CC(C=*)C2CC=CC12]

In certain embodiments, each instance of the first monomer is of Formula (D2), or a salt thereof. In certain embodiments, each instance of the first monomer is of Formula (D2).

In certain embodiments, each instance of x is 0. In certain embodiments, each instance of x is 1. In certain embodiments, each instance of x is 2.

In certain embodiments, each instance of y is 1. In certain embodiments, each instance of y is 0. In certain embodiments, each instance of v is 2.

In certain embodiments, each instance of x is 1, and each instance of y is 1. In certain embodiments, each instance of x is 1, and each instance of y is 0. In certain embodiments, each instance of x is 0, and each instance of y is 1.

In certain embodiments, each instance of the first monomer is of the formula:

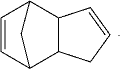

In certain embodiments, each instance of the first monomer is of the formula:

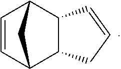

In certain embodiments, each instance of the first monomer is of the formula:

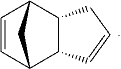

In certain embodiments, at least one instance of the first monomer is of the formula:

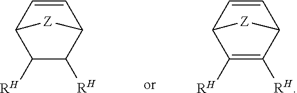

In certain embodiments, at least one instance of the first monomer is of the formula:

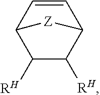

or salt thereof.

In certain embodiments, the hydroxylated oligomer or hydroxylated polymer, hydroxylated polymer, or copolymer is crosslinked. In certain embodiments, the hydroxylated oligomer or hydroxylated polymer is crosslinked because it comprises one or more instances of the crosslinking units. In certain embodiments, the crosslinking degree is between 5% and 50%, inclusive, mole:mole. In certain embodiments, the crosslinking degree is between 5% and 10%, inclusive, mole:mole. In certain embodiments, the crosslinking degree is between 10% and 20%, inclusive, mole:mole. In certain embodiments, the crosslinking degree is between 20% and 30%, inclusive, mole:mole. In certain embodiments, the crosslinking degree is between 30% and 40%, inclusive, mole:mole. In certain embodiments, the crosslinking degree is between 40% and 5(1%, inclusive, mole:mole. In certain embodiments, the crosslinking degree is not greater than the concentration of all the instances of the second in onotner in the hydroxylated oligomer or hydroxylated polymer, hydroxylated polymer, or copolymer, mole:mole. In certain embodiments, the hydroxylated polymer is a thermosetting polymer. In certain embodiments, the hydroxylated polymer is a thermosetting polymer. In certain embodiments, the copolymer is a thermosetting polymer.

In certain embodiments, the aqueous solubility of the hydroxylated oligomer or hydroxylated polymer is between 0.1 and 0.3, between 0.3 and 1, between 1 and 3, between 3 and 10, between 10 and 30, or between 30 and 100, inclusive, g/L, at 1 atmosphere and 20° C. In certain embodiments, the aqueous solubility of the hydroxylated oligomer or hydroxylated polymer is between 1 and 10, inclusive, g/L, at 1 atmosphere and 20° C.

In certain embodiments, the aqueous solubility of the hydroxylated polymer is between and 0.3, between 0.3 and 1, between 1 and 3, between 3 and 10, between 10 and 30, or between 30 and 100, inclusive, g/L, at 1 atmosphere and 20° C. In certain embodiments, the aqueous solubility of the hydroxylated polymer is between 1 and 10, inclusive, g/L, at 1 atmosphere and 20° C.

In certain embodiments, the molar ratio of the one or more instances of the first monomer to the one or more instances of the second monomer is between 100:1 and 30:1, between 30:1 and 10:1, between 10:1 and 3:1, or between 3:1 and 1:1, inclusive. In certain embodiments, the molar ratio of the one or more instances of the first monomer to the one or more instances of the second monomer is between 12:1 and 1.3:1, inclusive. In certain embodiments, the molar ratio of the one or more instances of the first monomer to the one or more instances of the second monomer is between 8:1 and 2:1, inclusive. In certain embodiments, the molar ratio of the one or more instances of the first monomer to the one or more instances of the second monomer is between 30:1 and 3:1, inclusive. In certain embodiments, the molar ratio of the one or more instances of the first monomer to the one or more instances of the second monomer is between 20:1 and 5:1, inclusive. In certain embodiments, the molar ratio of the one or more instances of the first monomer to the one or more instances of the second monomer is between 60:1 and 6:1, inclusive. In certain embodiments, the molar ratio of the one or more instances of the first monomer to the one or more instances of the second monomer is between 40:1 and 10:1, inclusive. In certain embodiments, the molar ratio of the one or more instances of the first monomer to one or more instances of a second monomer is between 1:2 and 2:1, inclusive, 6:1 and 19:1, inclusive, or 5:1 and 35:1, inclusive. In certain embodiments, the molar ratio of the one or more instances of the first monomer to the one or more instances of the second monomer is between 1:2 and 2:1, inclusive. In certain embodiments, the molar ratio of the one or more instances of the first monomer to the one or more instances of the second monomer is between 1:10 and 10:1 (e.g., between 1:5 and 5:1), inclusive. In some embodiments, the molar ratio of the one or more instances of the first monomer to the one or more instances of the second monomer is between 1:35 and 35:1, inclusive. In some embodiments, the molar ratio of the one or more instances of the second monomer to the one or more instances of the first monomer is between 1:33 and 1:27, inclusive. In some embodiments, the molar ratio of the one or more instances of the second monomer to the one or more instances of the first monomer is between 1:17 and 1:11, inclusive, In some embodiments, the molar ratio of the one or more instances of the second monomer to the one or more instances of the first monomer is between 1:11 and 1:6, inclusive. In certain embodiments, the molar ratio of the one or more instances of the first monomer to the one or more instances of the second monomer is about 1:1.

In certain embodiments, the volume ratio of the one or more instances of the first monomer to the one or more instances of the second monomer is between 100:1 and 30:1, between 30:1 and 10:1, between 10:1 and 3:1, or between 3:1 and 1:1, inclusive. In certain embodiments, the volume ratio of the one or more instances of the first monomer to the one or more instances of the second monomer is between 12:1 and 1.3:1. inclusive. In certain embodiments, the volume ratio of the one or more instances of the first monomer to the one or more instances of the second monomer is between 8:1 and 2:1, inclusive. In certain embodiments, the volume ratio of the one or more instances of the first monomer to the one or more instances of the second monomer is between 30:1 and 3:1, inclusive. In certain embodiments, the volume ratio of the one or more instances of the first monomer to the one or more instances of the second monomer is between 20:1 and 5:1, inclusive. In certain embodiments, the volume ratio of the one or more instances of the first monomer to the one or more instances of the second monomer is between 60:1 and 6:1, inclusive. In certain embodiments, the volume ratio of the one or more instances of the first monomer to the one or more instances of the second monomer is between 40:1 and 10:1, inclusive.

In certain embodiments, the average molecular weight of the hydroxylated. oligomer or hydroxylated polymer is between 300 Da and 1 kDa, between 1 kDa and 3 kDa, between 3 kDa and 10 kDa, between 10 kDa and 100 kDa, or between 100 kDa and 1,000 kDa, inclusive. In certain embodiments, the average molecular weight of the hydroxylated oligomer or hydroxylated polymer is between 1 kDa and 10 kDa, inclusive. In certain embodiments, the average molecular weight is as determined by gel permeation chromatography, in certain embodiments, the average molecular weight of the hydroxylated oligomer car hydroxylated polymer as determined by gel permeation chromatography is between 300 Da and 1,000 kDa, inclusive. in certain embodiments, the average molecular weight of the hydroxylated oligomer or hydroxylated polymer as determined by get permeation chromatography is between 1 kDa. and 8 kDa, inclusive.

In certain embodiments, the average molecular weight of the hydroxylated polymer is between 300 Da and 1 kDa, between 1 kDa, and 3 kDa, between 3 kDa. and 10 kDa, between 10 kDa and 100 kDa, or between 100 kDa and 1,000 kDa., inclusive. In certain embodiments, the average molecular weight of the hydroxylated polymer is between 1 kDa and 10 kDa, inclusive.

In certain embodiments, the average molecular weight is as determined by gel permeation chromatography. In certain embodiments, the average molecular weight of the hydroxylated polymer as determined by gel permeation chromatography is between 300 Da and 1,000 kDa, inclusive. In certain embodiments, the average molecular weight of the hydroxylated polymer as determined by gel permeation chromatography is between 1 kDa and 8 kDa, inclusive.

In certain embodiments, the average molecular weight of the copolymer is between 10 kDa and 10,000 kDa, inclusive. In certain embodiments, the average molecular weight of the copolymer is between 10 kDa, and 30 kDa, between 30 kDa, and 100 kDa, between 100 kDa and 1,000 kDa, between 1,000 kDa and 10,000 kDa, or between 10,000 kDa and 100,000 kDa, inclusive. In certain embodiments, the average molecular weight of the copolymer is between 10 kDa and 100 kDa, inclusive. In certain embodiments, the average molecular weight is as determined by gel permeation chromatography. In certain embodiments, the average molecular weight of the copolymer as determined by gel permeation chromatography is between 10 kDa, and 100,000 kDa, inclusive. In certain embodiments, the number average polymerization degree is between 2 and 1,000, inclusive, with respect to the first monomer; and between 2 and 1,000, inclusive, with respect to the second monomer. In certain embodiments, the number average polymerization degree is between 10 and 200, inclusive, with respect to the first monomer; and between 10 and 200, inclusive, with respect to the second monomer. In certain embodiments, the number average polymerization degree is between 15 and 100, inclusive, with respect to the first monomer; and between 15 and 100, inclusive, with respect to the second monomer. In certain embodiments, the number average polymerization degree is between 2 and 1,000, between 10 and 1,000, between 100 and 1,000, between 2 and 100, between 10 and 100, between 2 and 10, inclusive, with respect to the first monomer. In certain embodiments, the number average polymerization degree is between 2 and 1,000, between 10 and 1,000, between 100 and 1,000, between 2 and 100, between 10 and 100, between 2 and 10, inclusive, with respect to the second, monomer.

In certain embodiments, the dispersity (I)) of the copolymer is between 1 and 2, between 1.1 and 2, between 1.3 and 2, between 1.5 and 2, between 1.1 and 1.5. between 1.1 and 1.3, between 1.3 and 2, between 1.3 and 1.5, between 1.5 and 2, inclusive.

In certain embodiments, the average hydrodynamic diameter of the hydroxylated oligomer or hydroxylated polymer is between 1 and 100 nm, inclusive. In certain embodiments, the average hydrodynamic diameter of the hydroxylated oligomer or hydroxylated polymer is between 1 and 10 nm, inclusive. In certain embodiments, the average hydrodynamic diameter of the hydroxylated oligomer or hydroxylated polymer is between 10 and 30 nm, inclusive. In certain embodiments, the average hydrodynamic diameter of the hydroxylated oligomer or hydroxylated polymer is between 30 and 100 nm, inclusive. In certain embodiments, the average hydrodynamic diameter of the hydroxylated polymer is between 1 and 100 nm, inclusive. In certain embodiments, the average hydrodynamic diameter of the hydroxylated polymer is between 1 and 10 nm, inclusive. In certain embodiments, the average hydrodynamic diameter of the hydroxylated polymer is between 10 and 30 nm, inclusive. In certain embodiments, the average hydrodynamic diameter of the hydroxylated polymer is between 30 and 100 nm, inclusive. In certain embodiments, the average hydrodynamic diameter is as determined by diffusion ordered spectroscopy (DOSY).

In certain embodiments, the copolymer is a block copolymer, preferably a block polymer comprising at least four consecutive blocks, wherein:

each of the first consecutive block and the third consecutive block independently comprises one or more repeating units formed from the first monomer or the third monomer if present; and

each of the second consecutive block and the fourth consecutive block independently comprises one or more repeating units formed from the second monomer.

In certain embodiments, the copolymer is a random copolymer.

In certain embodiments, the step of polymerizing is substantially free (e.g., between 90%-99% free) of a chain transfer agent.

In certain embodiments, the step of hydrolyzing the copolymer comprises hydrolyzing at least 50% of the —O—Si bonds of the copolymer to form —OH. In certain embodiments, the step of hydrolyzing the copolymer comprises hydrolyzing between 50% and 70%, inclusive, of the —O—Si bonds of the copolymer to form —OH. In certain embodiments, the step of hydrolyzing the copolymer comprises hydrolyzing between 70% and 90%, inclusive, of the —O—Si bonds of the copolymer to form —OH. In certain embodiments, the step of hydrolyzing the copolymer comprises hydrolyzing between 90% and 99%, inclusive, of the —O—Si bonds of the copolymer to form —OH. In certain embodiments, the step of hydrolyzing the copolymer comprises hydrolyzing at least 95% of the —O—Si bonds of the copolymer to form —OH.

In certain embodiments, the step of hydrolyzing the copolymer comprises ambient temperature, ambient pressure, and a reaction time of between 1 hour and 48 hours (e.g., between 1 hour and 6 hours, between 6 hour and 24 hours, between 24 hour and 48 hours), inclusive.

In certain embodiments, the step of hydrolyzing the copolymer comprises reacting the copolymer with a fluoride source. In certain embodiments, the fluoride source is tetra(unsubstitutedalkyl)-ammonium fluoride. In certain embodiments, the fluoride source is tetra(unsubstituted C₁₋₆ alkyl)-ammonium fluoride (e.g., TBAF). In certain embodiments, the fluoride source is a metal fluoride (e.g., alkali metal fluoride or alkaline earth metal fluoride). In certain embodiments, a polymer is chemically degradable in the presence of tetra-n-butylammonium fluoride (TBAF). In certain embodiments, the fluoride source is an acidic fluoride source (e.g., HF). In certain embodiments, the fluoride source is a latent fluoride source (e.g., tris(dimethylamino)suffonium difluorotrimethyl silicate (TASF)).

In some embodiments, the amount of the fluoride source is about 1 equivalent (mole:mole) relative to the amount of the second monomer. In some embodiments, the amount of the fluoride source is in excess (e.g., about 2 equivalents) relative to the amount of the second monomer. In certain embodiments, the step of hydrolyzing the copolymer comprises reacting the copolymer with an acid.

In certain embodiments, the acid is an aqueous solution of an acid. In certain embodiments, the acid is an inorganic acid. In certain embodiments, the acid is an organic acid. In certain embodiments, the acid has a pKₐ value of less than 3, less than 2, less than 1, or less than 0, under ambient conditions, in certain embodiments, the acid is HCl, HBr, HI, HClO₄, HNO₃, H₂SO₄, CH₃SO₃H, or CF₃SO₃H. In certain embodiments, the acid is HCl. In certain embodiments, the acid is CF₃CO₂H.

In some embodiments, the amount of the acid is about 1 equivalent (mole:mole) relative to the amount of the second monomer. In some embodiments, the amount of the acid is in excess (e.g., about 2 equivalents) relative to the amount of the second monomer.

In another aspect, the present disclosure provides conjugates (conjugates of the present disclosure) prepared by reacting a hydroxy-reacting substance with a hydroxylated oligomer or hydroxylated polymer of the present disclosure, wherein hydroxy-reacting substance comprises at least one instance of a hydroxy-reacting moiety.

In another aspect, the present disclosure provides methods of preparing a conjugate comprising reacting a hydroxy-reacting substance with a hydroxylated oligomer or hydroxylated polymer of the present disclosure.

In certain embodiments, the hydroxy-reacting substance is a hydroxy-reacting small molecule. In certain embodiments, the hydroxy-reacting substance is a carboxylic acid, a carboxylic halide, a carboxylic anhydride, a sulfonic acid, a sulfonyl halide, a sulfonic anhydride, a sulfinic acid, a sulfinyl halide, or a sulfinic anhydride. In certain embodiments, the hydroxy-reacting substance is lactide. In certain embodiments, the hydroxy-reacting substance is a hydroxy-reacting polymer. In certain embodiments, the average molecular weight of the hydroxy-reacting polymer is between 1 kDa and 3 kDa, between 3 kDa and 10 kDa, between 10 kDa and 30 kDa, between 30 kDa and 100 kDa, or between 100 kDa and 1,000 kDa, inclusive. In certain embodiments, the average molecular weight of the hydroxy-reacting polymer is between 3 kDa and 30 kDa, inclusive, In certain embodiments. the average molecular weight is as determined by gel permeation chromatography. in certain embodiments, the average molecular weight of the hydroxy-reacting polymer as determined by gel permeation chromatography is between 1 kDa and 1,000 kDa, inclusive.

In certain embodiments, the hydroxy-reacting substance is a polysiloxane, wherein the polysiloxane comprises at least one instance of a hydroxy-reacting moiety. In certain embodiments, the hydroxy-reacting substance is a polydimethylsiloxane (PDMS), wherein the PDMS comprises at least one instance of a hydroxy-reacting moiety (e.g., hydride (e.g., Si(IV-H)).

In certain embodiments, at least one instance of the hydroxy-reacting moiety is Si(IV)-H, Si(IV)-(a leaving group), C(IV)-(a leaving group), —C(═O)—OH, —C(═O)-(a leaving group), —C(═O)—O—, —C(═O)—O—C(═O)—, —S)—O)—OH, —S(═O)-(a leaving group), —S(═O₂—OH, —S(═O)₂-(a leaving group, —OH, or —O-(a leaving grouip). In certain embodiments, at least one instance of the hydroxy-reacting moiety is Si(IV)-H. In certain embodiments, at least one instance of the hydroxy-reacting moiety is —C(═O)-(a leaving group). In certain embodiments, at least one instance of the hydroxy-reacting moiety is —O-(a leaving group).

In certain embodiments, the hydroxy-reacting substance is a polylactic acid (PLA). in certain embodiments, the hydroxy-reacting substance is a polyethylene glycol (PEG). In certain embodiments, the hydroxy-reacting substance is a PEG, wherein the average molecular weight of the PEG as determined by gel permeation chromatography is between 300 Da and I kDa., between 1 kDa and 3 kDa, between 3 kDa and 10 kDa, between 10 kDa and 30 kDa, between 30 kDa and 100 kDa, or between 100 kDa and 1,000 kDa, inclusive.

In another aspect, the present disclosure provides compositions (compositions of the present disclosure) comprising:

a compound, copolymer, hydroxylated oligomer, hydroxylated polymer, or conjugate of the present disclosure; and

optionally an excipient.

In certain embodiments, the composition of the present disclosure is a pharmaceutical composition. In certain embodiments, the composition of the present disclosure further comprises excipient. In certain embodiments, the pharmaceutical composition of the present disclosure further comprises a pharmaceutically acceptable excipient.

Compositions described herein can be prepared by any method known in the art. In general, such preparatory methods include bringing the hydroxylated polymer into association with an excipient, and/or one or more other accessory ingredients, and then, if necessary and/or desirable, shaping, and/or packaging the product into a desired unit.

In another aspect, the present disclosure provides kits comprising:

a compound, copolymer, hydroxylated oligomer, hydroxylated polymer, or conjugate of the present disclosure; and

instructions for using the compound, copolymer, hydroxylated oligomer, hydroxylated polymer, or conjugate.

Kits may be commercial packs or reagent packs. The kits may further comprise a container (e.g., a vial, ampule, bottle, syringe, and/or dispenser package, or other suitable container). In certain embodiments, a kit further comprises instructions for using the compound. In certain embodiments, a kit further comprises instructions for using the copolymer. In certain embodiments, a kit further comprises instructions for using the hydroxylated polymer (e.g., for preparing a conjugate). In certain embodiments, a kit further comprises instructions for using the conjugate. The details of certain embodiments of the invention are set forth in the present section.

Other features, objects, and advantages of the invention will be apparent from the Definitions, Figures, Examples, and Claims. The aspects described herein are not limited to specific embodiments, methods, apparati, or configurations, and as such can, of course, vary. The terminology used herein is for the purpose of describing particular aspects only and, unless specifically defined herein, is not intended to be limiting.

### EXANIPLES

In order that the invention described herein may be more fully understood, the following examples are set forth. The synthetic and biological examples described herein are offered to illustrate the present disclosure and are not to be construed in any way as limiting their scope.

### Example 1

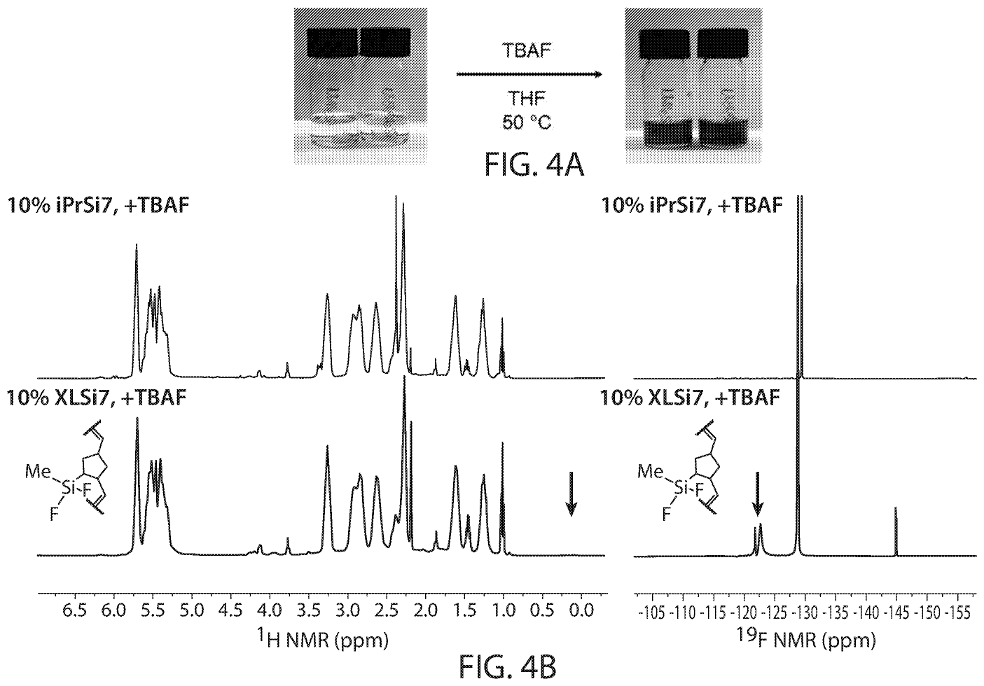

2.64 g of cis-butene diol (30 mmol) was dissolved in 1.5 L of dichloromethane. Next, 4.08 g of imidazole (60 mmol) was added. Finally, 30 mmol of dichlorosilane ((5-bicyclo[2.2.1]hept-2-enyl)methyldichlorosilane or [(5-bicyclo[2.2.1]hept-2-enypethyl]methyldichlorosilane) in 100 mL of DOA was added dropwise over 1 hour. A significant quantity of white precipitate formed. The solution was then filtered through a 2×2×2 in. pad of silica and concentrated to yield the corresponding XL monomer compounds as clear oils. The XI monomer prepared from 5-bicyclo[2.2.1]hept-2-enypmethyldichlorosilane was XLSi7. The XL monomer prepared from [(5-bicyclo[2.2,1]hept-2-enyl)ethyl]methyldichlorosilane was XISi7-2. Exemplary results are shown in FIG. 3.

### Example 2

100 μL of XL monomer was added to a 1 mL vial, followed by 900 μL of DCPD and mixed thoroughly by vortex. Separately, 2.0 mg of finely powdered Grubbs 2nd generation catalyst were weighed into a 1 mL vial. The DCPD/XL monomer mixture was added to the catalyst, vortexed until complete dissolution, and transferred in 150 μL aliquots to 1 mL vials. The samples were immediately cured at 120° C. for 30 minutes, then cooled to room temperature and removed form the vials with a hammer. The resulting copolymer was DCPD-XLSi7 or DCPD-XLSi7-2.

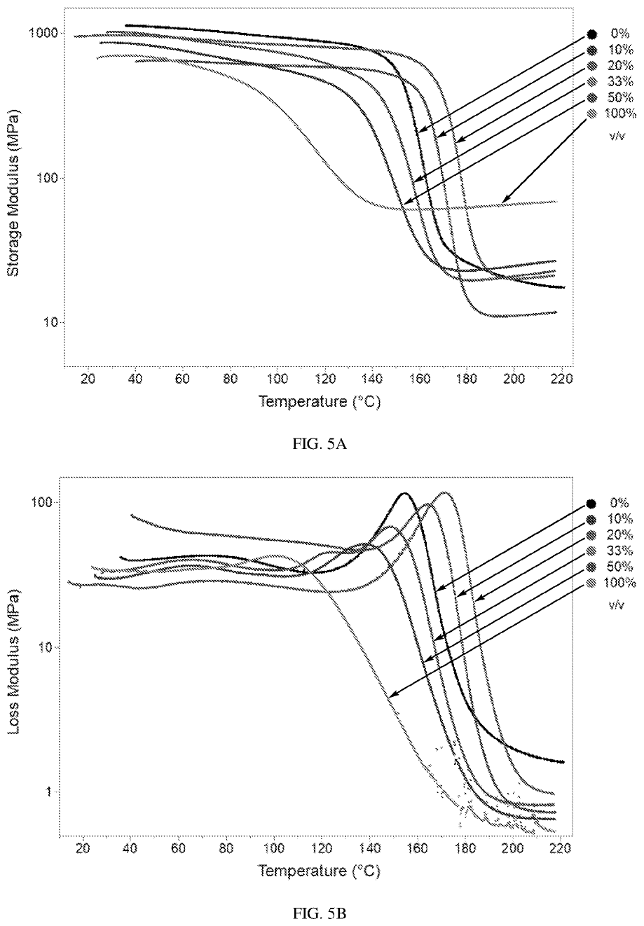

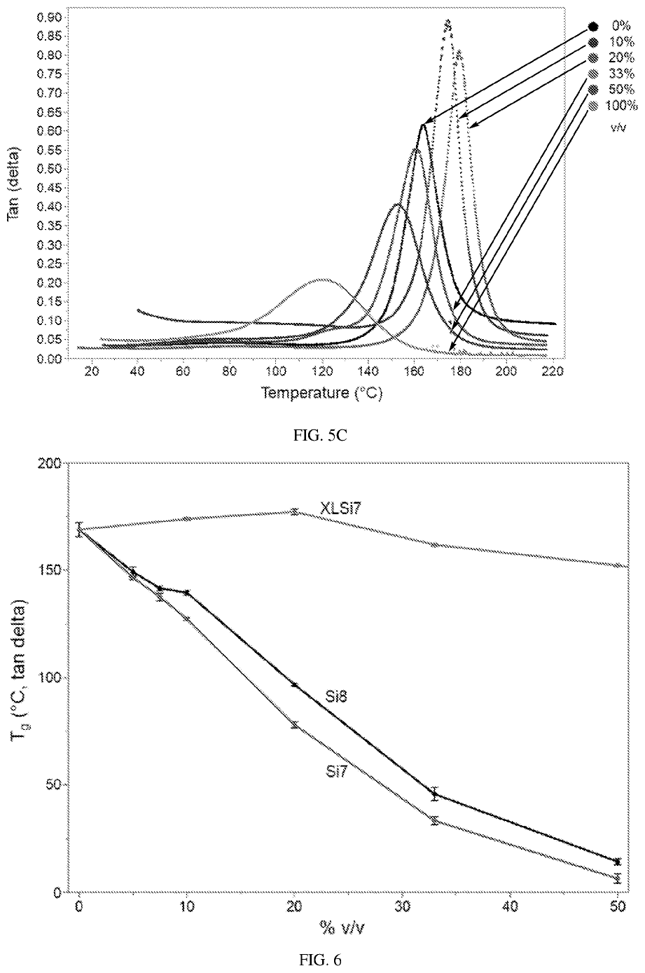

Samples were then placed in 5 mL of 0.2M tetrabutylammonium fluoride in THF, and heated to 50° C. on a hot plate overnight, resulting in full degradation of solids. Final solid pieces were removed from solution, and the solution was concentrated under vacuum to 1 mL, then slowly dropped into 100 mL acetone under rapid stirring. The resulting precipitate was filtered off, dissolved in CDCl3 and NMR spectra were taken. Exemplary results are shown in FIGS. 4A and 4B.

### Example 3

100 μL of XL monomer was added to a 1 mL vial, followed by 900 μL of DCPD and mixed thoroughly by vortex. Separately, 2.0 mg of finely powdered Grubbs 2nd generation catalyst were weighed into a 1 mL vial. The DCPD/XL monomer mixture was added to the catalyst, vortexed until complete dissolution, and transferred in ˜300 μL aliquots to rectangular silicon molds. The samples were immediately cured in the molds at 120° C. for 30 minutes, then cooled to room temperature and removed from the molds. The resulting copolymer was DCPD-XLSi7.

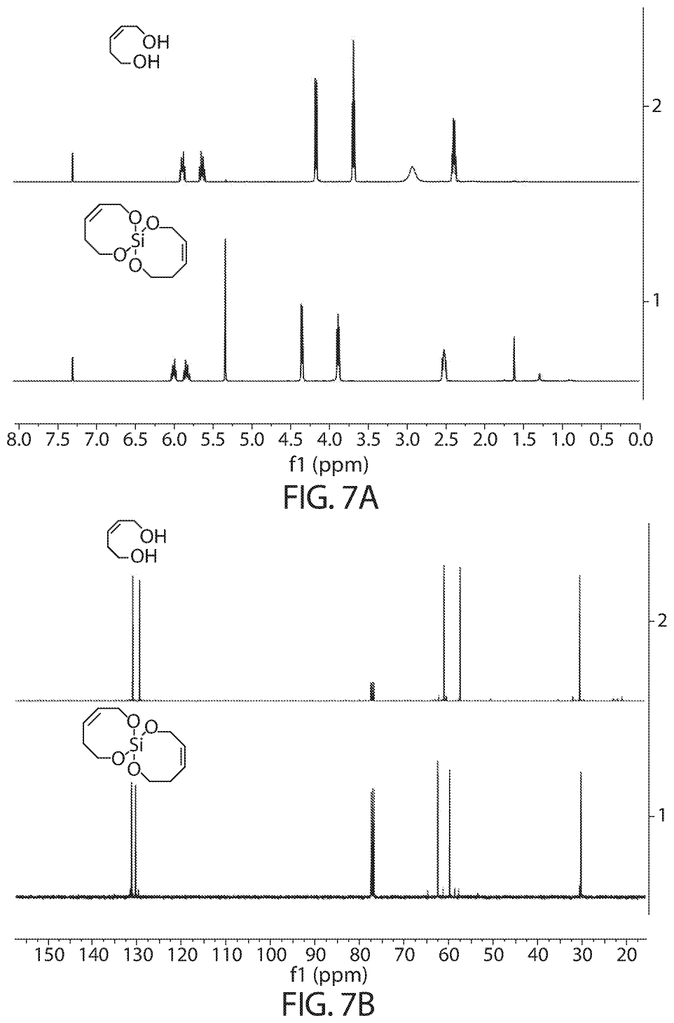

The samples were sanded down to have cross-sectional dimensions ˜2.5×3.0 mm, and 0 were analyzed by dynamic mechanical analysis in triplicate, in tensile mode using a TA Instruments DMAQ800. Samples were measured using 125% force tracking, 0.1N preload force and 10 μm amplitude strain at 1 Hz, from room temperature to 220° C. at a heating rate of 3° C. min ⁻¹. Exemplary results are shown in FIGS. 5A to 5C.

### Example 4

2.72 g of imidazole was dissolved in 1 L of dry DCM. To this solution was added 2.02 g of (Z)-pent-2-ene-1,5-diol and 1.69 g of SiCl₄, each dissolved in DCII to a final volume of 24 mL, over the course of two hours with a syringe pump. The resulting cloudy mixture was then filtered through a 2×2×2 in. plug of silica and concentrated to yield 150 mg of SpiroSi as a moisture-sensitive clear oil.

### Example 5

900 μL of DCPD was added to 100 μL of SpiroSi. The mixture was added to a vial containing 2 mg/mL of finely powdered Grubbs' 2nd generation catalyst The resulting mixture was added as 200 mg portions into glass vials, and heated at 120° C. for 30 minutes to cure. The vials were then broken to release the samples. Those samples were 10% v/v samples of SpiroSi-doped pDCPD.

An analogous protocol using 950 μL of DCPD and 50 μL of SpiroSi was used to synthesize 5% v/v samples of SpiroSi-doped pDCPD.

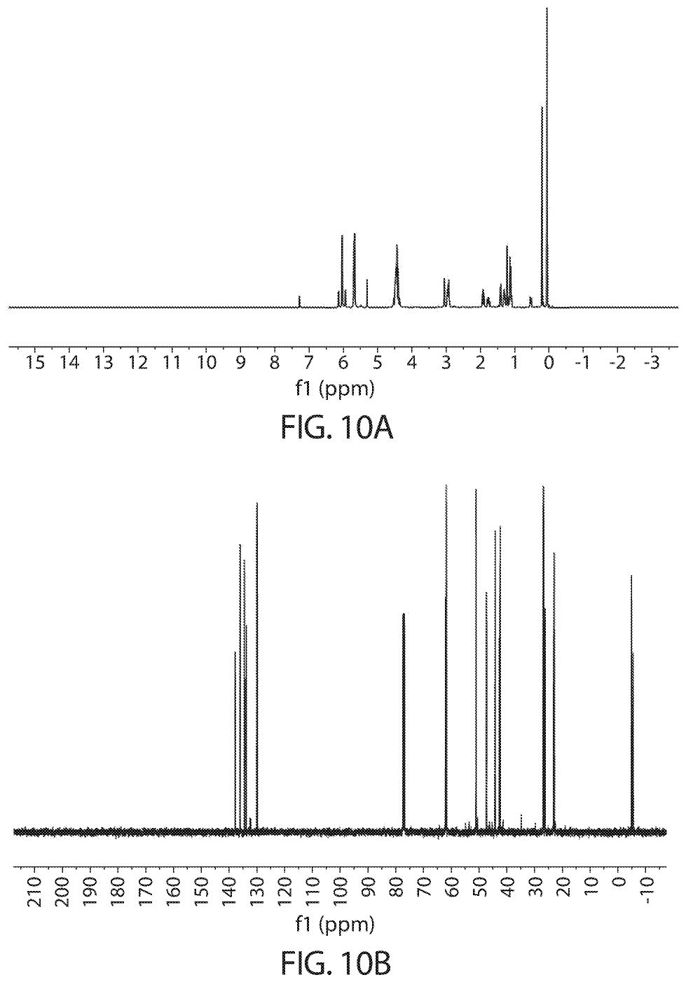

A sample was incubated with 5 mL of 0.2 M TBAF in THY for 24 hours. The soluble fragments were carefully removed by pipette and the residual solids were resuspended in fresh THF. The fragments were redissolved in chloroform, concentrated, and characterized by NMR. Exemplary results are shown in FIG. 8.

### Example 6

0.880 g of cis-butane-diol and 1.36 g of imidazole were dissolved in 1 L of dry DCM. Next, (5-bicycle[2.2.1]hept-2-enyl)methyldichlorosilane in 100 mL of DCM was added dropwise over 1 hour. The solution was then filtered through a 2×2×2 plug of silica and concentrated to yield 0.95 g of NbMeSi as a clear oil. Exemplary NMR results are shown in FIGS. 10A and 10B.

### Examples 7

1 mL of monomer solution was added to a vial containing 2 mg/mL of finely powdered Grubbs' 2nd generation catalyst. The resulting mixture was added into a silicone mold (approximately 300 μL per mold). The samples were heated at 120° C. for 30 minutes, then taken out of the oven and cut out of the mold. The samples were further sanded before measurement by Dynamic Mechanical Analysis (DMA).

DMA was conducted in tensile mode using a TA Instruments Q800. Temperature sweeps were conducted using 10 μm fixed amplitude, 0.01N preload force and 125.0% force tracking, with a frequency of 1 Hz and a heating rate of 3 degrees Celsius per minute, from room temperature to 220 Celsius.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context, The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g, in Markush group format, each subgroup of the elements is also disclosed, and any element(s) can be removed from the group. It should it be understood that, in general, where the invention, or aspects of the invention, is,/are referred to as comprising particular elements and/or features, certain embodiments of the invention or aspects of the invention consist, or consist essentially of, such elements and/or features. For purposes of simplicity, those embodiments have not been specifically set forth in hoer: verha herein. It is also noted that the terms “comprising,” “including,” and “containing,” and all other tenses thereof, are intended to be open and permits the inclusion of additional possibilities (e4;., elements or steps). Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (B): 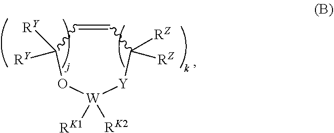
or a salt thereof, wherein:
W is carbon or silicon;
Y is O or C(R^{Q})₂;
each instance of R^{Q} is independently hydrogen, halogen, or substituted or unsubstituted, C₁₋₆ alkyl;
each instance of R^{Y} is independently hydrogen, halogen, or substituted or unsubstituted, C₁₋₆ alkyl;
each instance of R^{Z} is independently hydrogen, halogen, or substituted or unsubstituted, C₁₋₆ alkyl;
R^{K1} is hydrogen, halogen, substituted or unsubstituted, C₁₋₁₀ alkyl, substituted or unsubstituted, C₂₋₁₀ alkenyl, substituted or unsubstituted, C₂₋₁₀ alkynyl, substituted or unsubstituted carbocyclyl, substituted or unsubstituted heterocyclyl, substituted or unsubstituted aryl, substituted or unsubstituted heteroaryl, -L^{K1}-(substituted or unsubstituted carbocyclyl), -L^{K1}-(substituted or unsubstituted heterocyclyl), -L^{K1}-(substituted or unsubstituted aryl), -L^{K1}-(substituted or unsubstituted heteroaryl), or —OR^{N1};
L^{K1} is —O—, substituted or unsubstituted, C₁₋₁₀ alkylene, substituted or unsubstituted, C₂₋₁₀ heteroalkylene, substituted or unsubstituted carbocyclylene, substituted or unsubstituted heterocyclylene, substituted or unsubstituted arylene, substituted or unsubstituted heteroarylene, or a combination thereof;
R^{N1} is independently hydrogen, substituted or unsubstituted acyl, substituted or unsubstituted, C₁₋₁₀ alkyl, substituted or unsubstituted carbocyclyl, substituted or unsubstituted heterocyclyl, substituted or unsubstituted aryl, substituted or unsubstituted heteroaryl, or an oxygen protecting group;
R^{K2} is halogen, substituted or unsubstituted, C₂₋₁₀ alkenyl, substituted or unsubstituted C₂₋₁₀ alkynyl, substituted or unsubstituted carbocyclyl, substituted or unsubstituted heterocyclyl, substituted or unsubstituted heteroaryl, -L^{K2}-(substituted or unsubstituted carbocyclyl), -L^{K2}-(substituted or unsubstituted heterocyclyl), -L^{K2}-(substituted or unsubstituted aryl), or —OR^{N2}-;
L^{K2} is —O—, substituted or unsubstituted, C₁₋₁₀ alkylene, substituted or unsubstituted, C₂₋₁₀ heteroalkylene, substituted or unsubstituted carbocyclylene, substituted or unsubstituted heterocyclylene, substituted or unsubstituted arylene, substituted or unsubstituted heteroarylene, or a combination thereof;
R^{N2} is independently hydrogen, substituted or unsubstituted acyl, substituted or unsubstituted, C1-io alkyl, substituted or unsubstituted carbocyclyl, substituted or unsubstituted heterocyclyl, substituted or unsubstituted aryl, substituted or unsubstituted heteroaryl, or an oxygen protecting group;
or R^{K1} and R^{K2} are joined with the intervening atom to form substituted or unsubstituted carbocyclyl or substituted or unsubstituted heterocyclyl;
j is 1, 2, or 3; and
k is 1, 2, or 3;
provided that the compound is not of the formula:

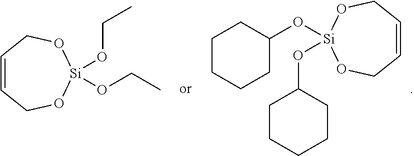 [SMILES: CCO[Si]1(OCC)OCC=CCO1 ; C1=CCO[Si](OC2CCCCC2)(OC2CCCCC2)OC1]

2. The compound of claim 1, or a salt thereof, provided that the compound is not of the formula:

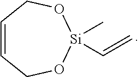

3-65. (canceled)

66. The compound of claim 1, or a salt thereof, wherein the compound is of the formula:

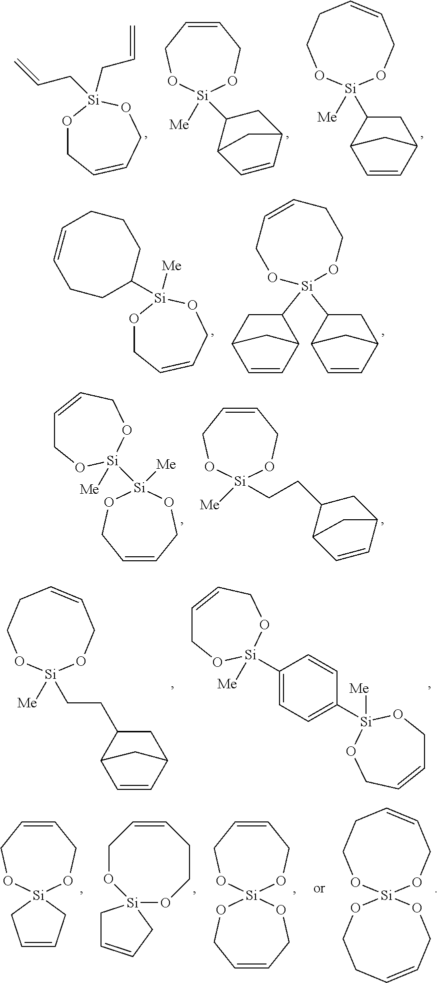

67. The compound of claim 1, or a salt thereof, wherein the compound is of the formula:

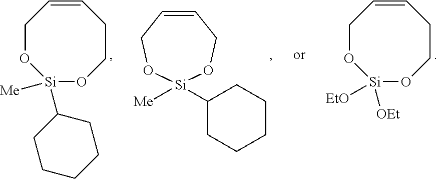

68. The compound of claim 1, or a salt thereof, wherein the compound is of the formula:

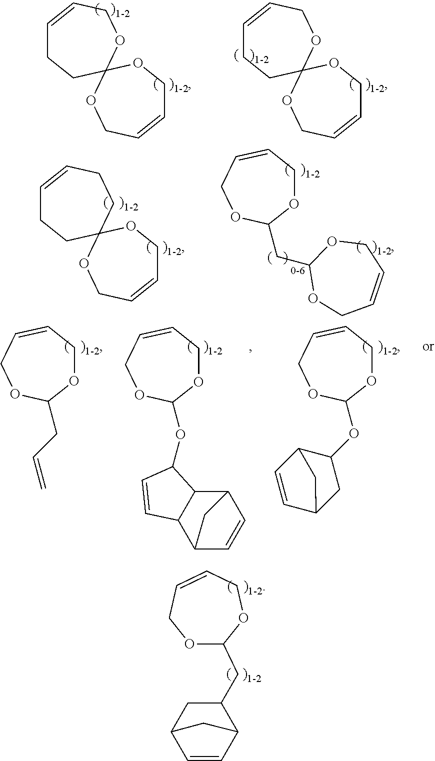

69. A copolymer prepared by a method comprising polymerizing:
one or more instances of a first monomer;
one or more instances of a second monomer, wherein the second monomer is a compound of claim 1, or a salt thereof; and
optionally one or more instances of a third monomer;
wherein any two instances of the first monomer are the same as or different from each other, any two instances of the second monomer are the same as or different from each other, any two instances of the third monomer are the same as or different from each other, and each instance of the first monomer, the second monomer, and the third monomer if present, is different from each other;
in the presence of a metathesis catalyst.

70. A method of preparing a copolymer of claim 69 comprising polymerizing:
one or more instances of a first monomer;
one or more instances of a second monomer, wherein the second monomer is a compound of claim 1, or a salt thereof; and
optionally one or more instances of a third monomer;
wherein any two instances of the first monomer are the same as or different from each other, any two instances of the second monomer are the same as or different from each other, any two instances of the third monomer are the same as or different from each other, and each instance of the first monomer, the second monomer, and the third monomer if present, is different from each other;
in the presence of a metathesis catalyst.

71. A hydroxylated oligomer or hydroxylated polymer prepared by a method comprising hydrolyzing a copolymer of claim 69, wherein the step of hydrolyzing the copolymer comprises hydrolyzing one or more instances of the —O—Si bonds of the copolymer to form —OH.

72. A method of preparing a hydroxylated oligomer or hydroxylated polymer comprising hydrolyzing a copolymer of claim 69, wherein the step of hydrolyzing the copolymer comprises hydrolyzing one or more instances of the —O—Si bonds of the copolymer to form —OH.

73-100. (canceled)

101. A conjugate prepared by reacting a hydroxy-reacting substance with a hydroxylated oligomer or hydroxylated polymer of claim 71, wherein hydroxy-reacting substance comprises at least one instance of a hydroxy-reacting moiety.

102. A method of preparing a conjugate comprising reacting a hydroxy-reacting substance with a hydroxylated oligomer or hydroxylated polymer of claim 71.

103-109. (canceled)

110. A composition comprising:
a compound of claim 1, of a salt thereof; and
optionally an excipient.

111. A kit comprising:
a compound of claim 1, or a salt thereof; and
instructions for using the compound or salt.

112. A composition comprising:
a copolymer of claim 69; and
optionally an excipient.

113. A composition comprising:
a hydroxylated oligomer or hydroxylated polymer of claim 71; and
optionally an excipient.

114. A composition comprising:
a conjugate of claim 101; and
optionally an excipient.

115. A kit comprising:
a copolymer of claim 69; and
instructions for using the copolymer.

116. A kit comprising:
a hydroxylated oligomer or hydroxylated polymer of claim 71; and
instructions for using the hydroxylated oligomer or hydroxylated polymer.

117. A kit comprising:
a conjugate of claim 101; and
instructions for using the conjugate.
